SEQUENCE LISTING (89 sequences)

SEQ ID NO: 1 - DNA, 5063 bp, Homo sapiens
gccatggagcccgagtgagcgcggcgcgggcccgtccggccgccggacaacatggaggcagcgccgcccgggccgccgtggccgctgctgctgctgctgctgctgctgctggcgctgtgcggctgcccggcccccgccgcggcctcgccgctcctgctatttgccaaccgccgggacgtacggctggtggacgccggcggagtcaagctggagtccaccatcgtggtcagcggcctggaggatgcggccgcagtggacttccagttttccaagggagccgtgtactggacagacgtgagcgaggaggccatcaagcagacctacctgaaccagacgggggccgccgtgcagaacgtggtcatctccggcctggtctctcccgacggcctcgcctgcgactgggtgggcaagaagctgtactggacggactcagagaccaaccgcatcgaggtggccaacctcaatggcacatcccggaaggtgctcttctggcaggaccttgaccagcctagggccatcgccttggaccccgctcacgggtacatgtactggacagactggggtgagacgccccggattgagcgggcagggatggatggcagcacccggaagatcattgtggactcggacatttactggcccaatggactgaccatcgacctggaggagcagaagctctactgggctgacgccaagctcagcttcatccaccgtgccaacctggacggctcgttccggcagaaggtggtggagggcagcctgacgcaccccttcgccctgacgctctccggggacactctgtactggacagactggcagacccgctccatccatgcctgcaacaagcgcactggggggaagaggaaggagatcctgagtgccctctactcacccatggacatccaggtgctgagccaggagcggcagcctttcttccacactcgctgtgaggaggacaatggcggctgctcccacctgtgcctgctgtccccaagcgagcctttctacacatgcgcctgccccacgggtgtgcagctgcaggacaacggcaggacgtgtaaggcaggagccgaggaggtgctgctgctggcccggcggacggacctacggaggatctcgctggacacgccggacttcaccgacatcgtgctgcaggtggacgacatccggcacgccattgccatcgactacgacccgctagagggctatgtctactggacagatgacgaggtgcgggccatccgcagggcgtacctggacgggtctggggcgcagacgctggtcaacaccgagatcaacgaccccgatggcatcgcggtcgactgggtggcccgaaacctctactggaccgacacgggcacggaccgcatcgaggtgacgcgcctcaacggcacctcccgcaagatcctggtgtcggaggacctggacgagccccgagccatcgcactgcaccccgtgatgggcctcatgtactggacagactggggagagaaccctaaaatcgagtgtgccaacttggatgggcaggagcggcgtgtgctggtcaatgcctccctcgggtggcccaacggcctggccctggacctgcaggaggggaagctctactggggagacgccaagacagacaagatcgaggtgatcaatgttgatgggacgaagaggcggaccctcctggaggacaagctcccgcacattttcgggttcacgctgctgggggacttcatctactggactgactggcagcgccgcagcatcgagcgggtgcacaaggtcaaggccagccgggacgtcatcattgaccagctgcccgacctgatggggctcaaagctgtgaatgtggccaaggtcgtcggaaccaacccgtgtgcggacaggaacggggggtgcagccacctgtgcttcttcacaccccacgcaacccggtgtggctgccccatcggcctggagctgctgagtgacatgaagacctgcatcgtgcctgaggccttcttggtcttcaccagcagagccgccatccacaggatctccctcgagaccaataacaacgacgtggccatcccgctcacgggcgtcaaggaggcctcagccctggactttgatgtgtccaacaaccacatctactggacagacgtcagcctgaagaccatcagccgcgccttcatgaacgggagctcggtggagcacgtggtggagtttggccttgactaccccgagggcatggccgttgactggatgggcaagaacctctactgggccgacactgggaccaacagaatcgaagtggcgcggctggacgggcagttccggcaagtcctcgtgtggagggacttggacaacccgaggtcgctggccctggatcccaccaagggctacatctactggaccgagtggggcggcaagccgaggatcgtgcgggccttcatggacgggaccaactgcatgacgctggtggacaaggtgggccgggccaacgacctcaccattgactacgctgaccagcgcctctactggaccgacctggacaccaacatgatcgagtcgtccaacatgctgggtcaggagcgggtcgtgattgccgacgatctcccgcacccgttcggtctgacgcagtacagcgattatatctactggacagactggaatctgcacagcattgagcgggccgacaagactagcggccggaaccgcaccctcatccagggccacctggacttcgtgatggacatcctggtgttccactcctcccgccaggatggcctcaatgactgtatgcacaacaacgggcagtgtgggcagctgtgccttgccatccccggcggccaccgctgcggctgcgcctcacactacaccctggaccccagcagccgcaactgcagcccgcccaccaccttcttgctgttcagccagaaatctgccatcagtcggatgatcccggacgaccagcacagcccggatctcatcctgcccctgcatggactgaggaacgtcaaagccatcgactatgacccactggacaagttcatctactgggtggatgggcgccagaacatcaagcgagccaaggacgacgggacccagccctttgttttgacctctctgagccaaggccaaaacccagacaggcagccccacgacctcagcatcgacatctacagccggacactgttctggacgtgcgaggccaccaataccatcaacgtccacaggctgagcggggaagccatgggggtggtgctgcgtggggaccgcgacaagcccagggccatcgtcgtcaacgcggagcgagggtacctgtacttcaccaacatgcaggaccgggcagccaagatcgaacgcgcagccctggacggcaccgagcgcgaggtcctcttcaccaccggcctcatccgccctgtggccctggtggtggacaacacactgggcaagctgttctgggtggacgcggacctgaagcgcattgagagctgtgacctgtcaggggccaaccgcctgaccctggaggacgccaacatcgtgcagcctctgggcctgaccatccttggcaagcatctctactggatcgaccgccagcagcagatgatcgagcgtgtggagaagaccaccggggacaagcggactcgcatccagggccgtgtcgcccacctcactggcatccatgcagtggaggaagtcagcctggaggagttctcagcccacccatgtgcccgtgacaatggtggctgctcccacatctgtattgccaagggtgatgggacaccacggtgctcatgcccagtccacctcgtgctcctgcagaacctgctgacctgtggagagccgcccacctgctccccggaccagtttgcatgtgccacaggggagatcgactgtatccccggggcctggcgctgtgacggctttcccgagtgcgatgaccagagcgacgaggagggctgccccgtgtgctccgccgcccagttcccctgcgcgcggggtcagtgtgtggacctgcgcctgcgctgcgacggcgaggcagactgtcaggaccgctcagacgaggcggactgtgacgccatctgcctgcccaaccagttccggtgtgcgagcggccagtgtgtcctcatcaaacagcagtgcgactccttccccgactgtatcgacggctccgacgagctcatgtgtgaaatcaccaagccgccctcagacgacagcccggcccacagcagtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctgtacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgcgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctgacctcggccgggccactctggcttctctgtgcccctgtaaatagttttaaatatgaacaaagaaaaaaatatattttatgatttaaaaaataaatataattgggattttaaaaacatgagaaatgtgaactgtgatggggtgggcagggctgggagaactttgta

SEQ ID NO: 2 - protein, 1615 aa, Homo sapiens
MEAAPPGPPWPLLLLLLLLLALCGCPAPAAASPLLLFANRRDVRLVDAGGVKLESTIVVSGLEDAAAVDFQFSKGAVYWTDVSEEAIKQTYLNQTGAAVQNVVISGLVSPDGLACDWVGKKLYWTDSETNRIEVANLNGTSRKVLFWQDLDQPRAIALDPAHGYMYWTDWGETPRIERAGMDGSTRKIIVDSDIYWPNGLTIDLEEQKLYWADAKLSFIHRANLDGSFRQKVVEGSLTHPFALTLSGDTLYWTDWQTRSIHACNKRTGGKRKEILSALYSPMDIQVLSQERQPFFHTRCEEDNGGCSHLCLLSPSEPFYTCACPTGVQLQDNGRTCKAGAEEVLLLARRTDLRRISLDTPDFTDIVLQVDDIRHAIAIDYDPLEGYVYWTDDEVRAIRRAYLDGSGAQTLVNTEINDPDGIAVDWVARNLYWTDTGTDRIEVTRLNGTSRKILVSEDLDEPRAIALHPVMGLMYWTDWGENPKIECANLDGQERRVLVNASLGWPNGLALDLQEGKLYWGDAKTDKIEVINVDGTKRRTLLEDKLPHIFGFTLLGDFIYWTDWQRRSIERVHKVKASRDVIIDQLPDLMGLKAVNVAKVVGTNPCADRNGGCSHLCFFTPHATRCGCPIGLELLSDMKTCIVPEAFLVFTSRAAIHRISLETNNNDVAIPLTGVKEASALDFDVSNNHIYWTDVSLKTISRAFMNGSSVEHVVEFGLDYPEGMAVDWMGKNLYWADTGTNRIEVARLDGQFRQVLVWRDLDNPRSLALDPTKGYIYWTEWGGKPRIVRAFMDGTNCMTLVDKVGRANDLTIDYADQRLYWTDLDTNMIESSNMLGQERVVIADDLPHPFGLTQYSDYIYWTDWNLHSIERADKTSGRNRTLIQGHLDFVMDILVFHSSRQDGLNDCMHNNGQCGQLCLAIPGGHRCGCASHYTLDPSSRNCSPPTTFLLFSQKSAISRMIPDDQHSPDLILPLHGLRNVKAIDYDPLDKFIYWVDGRQNIKRAKDDGTQPFVLTSLSQGQNPDRQPHDLSIDIYSRTLFWTCEATNTINVHRLSGEAMGVVLRGDRDKPRAIVVNAERGYLYFTNMQDRAAKIERAALDGTEREVLFTTGLIRPVALVVDNTLGKLFWVDADLKRIESCDLSGANRLTLEDANIVQPLGLTILGKHLYWIDRQQQMIERVEKTTGDKRTRIQGRVAHLTGIHAVEEVSLEEFSAHPCARDNGGCSHICIAKGDGTPRCSCPVHLVLLQNLLTCGEPPTCSPDQFACATGEIDCIPGAWRCDGFPECDDQSDEEGCPVCSAAQFPCARGQCVDLRLRCDGEADCQDRSDEADCDAICLPNQFRCASGQCVLIKQQCDSFPDCIDGSDELMCEITKPPSDDSPAHSSAIGPVIGIILSLFVMGGVYFVCQRVVCQRYAGANGPFPHEYVSGTPHVPLNFIAPGGSQHGPFTGIACGKSMMSSVSLMGGRGGVPLYDRNHVTGASSSSSSSTKATLYPPILNPPPSPATDPSLYNMDMFYSSNIPATARPYRPYIIRGMAPPTTPCSTDVCDSDYSASRWKASKYYLDLNSDSDPYPPPPTPHSQYLSAEDSCPPSPATERSYFHLFPPPPSPCTDSS

SEQ ID NO: 3 - DNA, 20 bp, Artificial Sequence
  Primer
ttgctgccctagacttagcc

SEQ ID NO: 4 - DNA, 18 bp, Artificial Sequence
  Primer
ccaagtcgcttccgagac

SEQ ID NO: 5 - DNA, 20 bp, Artificial Sequence
  Primer
catcccagggctgtgtatct

SEQ ID NO: 6 - DNA, 20 bp, Artificial Sequence
  Primer
acttgggctcatgcaaattc

SEQ ID NO: 7 - DNA, 20 bp, Artificial Sequence
  Primer
ccgatgggtgagattttagg

SEQ ID NO: 8 - DNA, 19 bp, Artificial Sequence
  Primer
cgtgggtacctaccggaac

SEQ ID NO: 9 - DNA, 20 bp, Artificial Sequence
  Primer
taattgggtcagcagcaatg

SEQ ID NO: 10 - DNA, 18 bp, Artificial Sequence
  Primer
gcactcacagaaaggctg

SEQ ID NO: 11 - DNA, 20 bp, Artificial Sequence
  Primer
agtgacggtcctcttctgga

SEQ ID NO: 12 - DNA, 20 bp, Artificial Sequence
  Primer
caagtggatcatttcgaacg

SEQ ID NO: 13 - DNA, 20 bp, Artificial Sequence
  Primer
tggctgagtatttcccttgc

SEQ ID NO: 14 - DNA, 20 bp, Artificial Sequence
  Primer
ccagaatgacaggtccaggt

SEQ ID NO: 15 - DNA, 20 bp, Artificial Sequence
  Primer
tgcttcttctccagcctcat

SEQ ID NO: 16 - DNA, 20 bp, Artificial Sequence
  Primer
atgtggccaaatagcagagc

SEQ ID NO: 17 - DNA, 20 bp, Artificial Sequence
  Primer
gcattgaacccgtcttgttt

SEQ ID NO: 18 - DNA, 20 bp, Artificial Sequence
  Primer
ggcacctgagctcaacactt

SEQ ID NO: 19 - DNA, 19 bp, Artificial Sequence
  Primer
tgctgggctgttgtgttta

SEQ ID NO: 20 - DNA, 20 bp, Artificial Sequence
  Primer
ctttgaggcaggaacagagg

SEQ ID NO: 21 - DNA, 20 bp, Artificial Sequence
  Primer
agcgaaactccgtctcaaaa

SEQ ID NO: 22 - DNA, 20 bp, Artificial Sequence
  Primer
gctctaatcactgagggcca

SEQ ID NO: 23 - DNA, 20 bp, Artificial Sequence
  Primer
gagggctgagctgaagaggt

SEQ ID NO: 24 - DNA, 19 bp, Artificial Sequence
  Primer
caggttggggaacttgcag

SEQ ID NO: 25 - DNA, 20 bp, Artificial Sequence
  Primer
attcatgtggtcgctaggct

SEQ ID NO: 26 - DNA, 20 bp, Artificial Sequence
  Primer
gaagctcctttcagcgtcag

SEQ ID NO: 27 - DNA, 20 bp, Artificial Sequence
  Primer
ccagctcctctgtggcttac

SEQ ID NO: 28 - DNA, 20 bp, Artificial Sequence
  Primer
tcctccctctgctaaggaca

SEQ ID NO: 29 - DNA, 19 bp, Artificial Sequence
  Primer
cagagctctccagccagtg

SEQ ID NO: 30 - DNA, 19 bp, Artificial Sequence
  Primer
ctgtgagaggctggcattc

SEQ ID NO: 31 - DNA, 19 bp, Artificial Sequence
  Primer
atgtgacctgtcagcctcg

SEQ ID NO: 32 - DNA, 21 bp, Artificial Sequence
  Primer
tgctgccattactgacaatga

SEQ ID NO: 33 - DNA, 20 bp, Artificial Sequence
  Primer
tctgtcctcccaagctgagt

SEQ ID NO: 34 - DNA, 20 bp, Artificial Sequence
  Primer
cacacaggatcttgcactgg

SEQ ID NO: 35 - DNA, 20 bp, Artificial Sequence
  Primer
catgagttctcatttggccc

SEQ ID NO: 36 - DNA, 20 bp, Artificial Sequence
  Primer
gccacagggactgtgatttt

SEQ ID NO: 37 - DNA, 20 bp, Artificial Sequence
  Primer
caacttctgctttgaagccc

SEQ ID NO: 38 - DNA, 20 bp, Artificial Sequence
  Primer
cagagcccctactcctgtga

SEQ ID NO: 39 - DNA, 19 bp, Artificial Sequence
  Primer
ccagaccttggttgctgtg

SEQ ID NO: 40 - DNA, 20 bp, Artificial Sequence
  Primer
cgtctcctcccctaaactcc

SEQ ID NO: 41 - DNA, 20 bp, Artificial Sequence
  Primer
atgttggccacctctttctg

SEQ ID NO: 42 - DNA, 20 bp, Artificial Sequence
  Primer
ctgcctcctccagatcattc

SEQ ID NO: 43 - DNA, 20 bp, Artificial Sequence
  Primer
gagtctcgtgggtagtggga

SEQ ID NO: 44 - DNA, 20 bp, Artificial Sequence
  Primer
agaaagcaagcatgcctcag

SEQ ID NO: 45 - DNA, 20 bp, Artificial Sequence
  Primer
agccctctctgcaaggaaag

SEQ ID NO: 46 - DNA, 20 bp, Artificial Sequence
  Primer
gcccactagcacccagaata

SEQ ID NO: 47 - DNA, 18 bp, Artificial Sequence
  Primer
gacaggcctttcccgttc

SEQ ID NO: 48 - DNA, 20 bp, Artificial Sequence
  Primer
caggaggactctcatggtgg

SEQ ID NO: 49 - DNA, 20 bp, Artificial Sequence
  Primer
ttcgtcatgggtggtgtcta

SEQ ID NO: 50 - DNA, 20 bp, Artificial Sequence
  Primer
ttcctcgaatgatgtagggc

SEQ ID NO: 51 - DNA, 20 bp, Artificial Sequence
  Primer
acctggacttcgtgatggac

SEQ ID NO: 52 - DNA, 20 bp, Artificial Sequence
  Primer
cagaacagtgtccggctgta

SEQ ID NO: 53 - DNA, 18 bp, Artificial Sequence
  Primer
ccatggagcccgagtgag

SEQ ID NO: 54 - DNA, 20 bp, Artificial Sequence
  Primer
gtcaaggtcctgccagaaga

SEQ ID NO: 55 - DNA, 20 bp, Artificial Sequence
  Primer
gggcaagaagctgtactgga

SEQ ID NO: 56 - DNA, 20 bp, Artificial Sequence
  Primer
tggatgtccatgggtgagta

SEQ ID NO: 57 - DNA, 18 bp, Artificial Sequence
  Primer
cagacccgctccatccat

SEQ ID NO: 58 - DNA, 20 bp, Artificial Sequence
  Primer
tcgttgatctcggtgttgac

SEQ ID NO: 59 - DNA, 20 bp, Artificial Sequence
  Primer
atcgactacgacccgctaga

SEQ ID NO: 60 - DNA, 20 bp, Artificial Sequence
  Primer
gtagatgaagtcccccagca

SEQ ID NO: 61 - DNA, 21 bp, Artificial Sequence
  Primer
gccaagacagacaagatcgag

SEQ ID NO: 62 - DNA, 20 bp, Artificial Sequence
  Primer
tgtggttgttggacacatca

SEQ ID NO: 63 - DNA, 20 bp, Artificial Sequence
  Primer
cacaggatctccctcgagac

SEQ ID NO: 64 - DNA, 20 bp, Artificial Sequence
  Primer
ctcgatcatgttggtgtcca

SEQ ID NO: 65 - DNA, 20 bp, Artificial Sequence
  Primer
cagccctttgttttgacctc

SEQ ID NO: 66 - DNA, 20 bp, Artificial Sequence
  Primer
tccagtagagatgcttgcca

SEQ ID NO: 67 - DNA, 19 bp, Artificial Sequence
  Primer
aagcgcattgagagctgtg

SEQ ID NO: 68 - DNA, 18 bp, Artificial Sequence
  Primer
ctcctcgtcgctctggtc

SEQ ID NO: 69 - DNA, 21 bp, Artificial Sequence
  Primer
cacaggggagatcgactgtat

SEQ ID NO: 70 - DNA, 19 bp, Artificial Sequence
  Primer
acatactcgtgcgggaagg

SEQ ID NO: 71 - DNA, 18 bp, Artificial Sequence
  Primer
gtccagcagctcgtccag

SEQ ID NO: 72 - DNA, 20 bp, Artificial Sequence
  Primer
tacaaagttctcccagccct

SEQ ID NO: 73 - DNA, 18 bp, Artificial Sequence
  Primer
tcatggacgggaccaact

SEQ ID NO: 74 - DNA, 19 bp, Artificial Sequence
  Primer
ggtgtagtgtgaggcgcag

SEQ ID NO: 75 - protein, 13 aa, Artificial Sequence
  Peptide that can act as an effector of BSMR
RVRLASHLRKLRK

SEQ ID NO: 76 - protein, 11 aa, Artificial Sequence
  Peptide that can act as an effector of BSMR
RLTRKRGLKLA

SEQ ID NO: 77 - protein, 11 aa, Artificial Sequence
  Peptide that can act as an effector of BSMR
CRAKRNNFKSA

SEQ ID NO: 78 - protein, 5 aa, Artificial Sequence
  Peptide that can act as an effector of BSMR
LKWKS

SEQ ID NO: 79 - protein, 24 aa, Artificial Sequence
  Peptide that can act as an effector of BSMR
KIRVKAGETQKKVIFCSREKVSHL

SEQ ID NO: 80 - protein, 27 aa, Artificial Sequence
  Peptide that can act as an effector of BSMR
FIPLKPTVKMLERSNHVSRTEVSSNHV

SEQ ID NO: 81 - protein, 20 aa, Artificial Sequence
  Peptide that can act as an effector of BSMR
DKGMAPALRHLYKELMGPWN

SEQ ID NO: 82 - protein, 14 aa, Artificial Sequence
  Peptide that can act as an effector of BSMR
DALKLAIDNALSIT

SEQ ID NO: 83 - protein, 17 aa, Artificial Sequence
  Peptide that can act as an effector of BSMR
RVRLASHLRKLRKRLLR

SEQ ID NO: 84 - DNA, 24 bp, Artificial Sequence
  Sequence encoding a FLAG antibody epitope
gactacaaggacgacgatgacaag

SEQ ID NO: 85 - DNA, 45 bp, Artificial Sequence
  Primer
gactacaaggacgacgatgacaagaccatcgtggtcagcggcctg

SEQ ID NO: 86 - DNA, 42 bp, Artificial Sequence
  Primer
cttgtcatcgtcgtccttgtaggactccagcttgactccgcc

SEQ ID NO: 87 - DNA, 30 bp, Artificial Sequence
  Sequence encoding an MYC antibody epitope
gagcagaagctgatatccgaggaggacctg

SEQ ID NO: 88 - DNA, 45 bp, Artificial Sequence
  Primer
gagcagaagctgatatccgaggaggacctgtgacctcggccgggc

SEQ ID NO: 89 - DNA, 45 bp, Artificial Sequence
  Primer
caggtcctcctcggatatcagcttctgctcggatgagtccgtgca

(19) United States
(12) Patent Application Publication
(10) Pub. No.: US 2003/0027151 A1
(43) Pub. Date: Feb. 6, 2003

(54) Regulator gene and system useful for the diagnosis and therapy of osteoporosis

(76) Inventors: Matthew L. Warman, Shaker Heights, OH (US); Yaoqin Gong, Jinan (CN); Bjorn R. Olsen, Milton, MA (US); Georges Rawadi, Paris (FR); Sergio Roman-Roman, Paris (FR)

Correspondence Address: HELLER EHRMAN WHITE & MCAULIFFE LLP, 1666 K STREET,NW, SUITE 300, WASHINGTON, DC, 20006, US

(21) Appl. No.: 09/931,375

(22) Filed: Aug. 17, 2001

Related U.S. Application Data
(60) Provisional application No. 60/304,851, filed on Jul. 13, 2001.
(60) Provisional application No. 60/226,119, filed on Aug. 18, 2000.
(60) Provisional application No. 60/234,337, filed on Sep. 22, 2000.

(51) Int. Cl. C12Q 1/68; C07H 21/04

(52) U.S. Cl. 435/6; 536/23.2

(57) ABSTRACT

A bone strength and mineralization regulatory (“BSMR”) protein is provided that can exist in multiple forms and that affects bone density. Polymorphic gene sequences of the protein are provided that are diagnostic of predipostion to osteoporosis. Other detection tools, compositions and methods of their use also are provided for predicting, evaluating and altering bone strength and mineralization status. The invention provides new natural and synthetic pharmaceuticals that effect the BSMR regulatory pathway and improve bone status. Tools also are provided for finding new pharmaceuticals that operate by binding to BSMR and that activate and/or deactivate this protein's biological function related to osteoporosis and blood vessel formation.

This application claims priority to U.S. Provisional Application No. 60/304,851, filed Jul. 13, 2001; U.S. Provisional Application No. 60/226,119, filed Aug. 18, 2000; and U.S. Provisional Application No. 60/234,337, filed Sep. 22, 2000.

### FIELD OF THE INVENTION

The invention relates generally to polynucleic acid and polypeptide sequences associated with regulation of bone strength and mineralization and more particularly to the use of polynucleic acid, peptides, pharmaceuticals and antibodies for the diagnosis and regulation of bone strength and mineralization.

### BACKGROUND OF THE INVENTION

Osteoporosis is a common medical problem with major morbidity and societal cost. Individuals afflicted with this disease present diminished bone strength as a consequence of low bone mineral content. Heritable factors have been implicated as causes of osteoporosis. One osteoporosis disease that has a genetic cause is osteoporosis-pseudoglioma syndrome (“OPS”), which is characterized by severe juvenile-onset osteoporosis and congenital or juvenile-onset blindness. Because of the postulated mendelian genetic basis of this disease, patients and their families that present OPS symptoms have been studied to determine the location of the responsible gene. An early report from such studies indicated that the gene may reside within human chromosome 11 Gong et al., Am. J. Hum. Gen. 55 suppl: A186 (1994). A more detailed report of this localization was published subsequently by Gong et al., Amer. J. Hum. Gen. 59: 146-151 (1996). In the years since that publication, the Warman laboratory intensively has looked for further clues to the disease by studying more affected patients and their families. However data from the additional families did not suffice for a more precise localization to an interval on human chromosome 11q13.

Within chromosome 11q13 is a sixth centiMorgan interval that contains an estimated 120 genes. Of these genes, a large number code proteins that directly or indirectly affect bone metabolism, as shown in FIG. 1 and as described, for example by Levanon et al., Genomics 7: 65-74, 1990. FIG. 1 is a partial list of genes that have been mapped within this interval on chromosome 11q13. Genes in this list encode proteins with putative roles in bone metabolism. Some of these genes are known to co-localize specifically with OPS. Each gene that co-localizes is a known potential candidate for causing OPS and has been labelled with an asterisk to denote this fact. Many of these genes are expressed in bone cells. For example, Chen et al. Genomics 55: 314-321 (1999) have studied a number of proteins that are expressed in bone cells and which seem to be coded by genes in this region.

Other genes within this interval have been studied, as for example explained by Mathew, et al., Oncogene 8: 191-193 (1993), Courseaux et al., Genomics 37: 354-365 (1996), Katsanis et al., Hum. Genet. 106: 66-72 (2000), Matsuo et al., Nature Genet. 24: 184-187 (2000), Grimmond et al., Genome Res. 6:124-131 (1996), Giguere et al., Nature 331:, 91-94 (1988), Guida et al., Mamm. Genome 9:240-2 (1998), Hey et al., Gene 216: 103-111 (1998), Chen et al., Genomics 55:314-321 (1999), Li et al., Nature Genet. 23: 447-451 (1999), Yin et al., J. Biol. Chem. 270:10147-10160 (1995), Xie et al., Cytokine 11: 729-35 (1999) and Leonard et al., Biochem J. 347 Pt 3: 719-24 (2000). Thus, a large number of genes exist in the region of DNA that has been linked with OPS and many of these have discovered functions, which often relate to bone metabolism. However, prior to the present invention no specific evidence has linked OPS to a single gene product within this interval and no gene has been identified as causing this disease. Still further, there has been no sufficient teaching in the patent or scientific literatures of what effectors might be useful for regulating such a single gene, assuming that the gene existed and was understood.

In sum, despite significant research effort, the medical research community has not determined the biochemical defect which causes OPS and efforts to clone genes known to cause the disease have been unsuccessful. This lack of information is unfortunate because further knowledge of a genetic and biochemical mechanism involved in bone strength and mineralization might provide clues to new methods and compositions for increasing bone strength and mineralization in patients having sub-optimum bone strength for reasons other than genetic disease.

### SUMMARY OF THE INVENTION

Accordingly, an object of this invention is to identify a DNA sequence coding for a regulatory protein in normal human cells that affects bone strength and mineralization and to provide a human gene sequence which can be used to improve bone status of diseased and of normal humans.

In accordance with an object of the invention one embodiment of the invention is an isolated DNA molecule useful for the diagnosis or therapy of osteoporosis, selected from the group consisting of a DNA molecule having a nucleotide sequence encoding an amino acid sequence as shown in FIG. 2; a DNA molecule capable of specific hybridization under stringent conditions to a DNA molecule according to the first member of the group, and which encodes a protein that, when introduced into human osteoblasts, increases the ability of the osteoblasts to synthesize bone; a DNA molecule having a nucleotide sequence which is degenerate as a result of the genetic code to the encoded protein amino acid sequence according to the second member of the group and which encodes a protein that, when introduced into osteoblast cells of humans, increases the ability of the cells to become osteoblasts and/or synthesize bone; a DNA molecule capable of specific hybridization under stringent conditions to a DNA molecule according to the first member, and which encodes a protein that, when introduced into epithelial cells of humans, increases the ability of the epithelial cells to form vascular tissue; and a DNA molecule having a nucleotide sequence which is degenerate as a result of the genetic code to the encoded protein amino acid sequence according to the fourth member of the group, and which encodes a protein that, when introduced into epithelial cells of humans, increases the ability of the epithelial cells to form vascular tissue. Other embodiments of the invention use such a DNA molecule within an expression vector that codes for a transmembrane protein that modulates bone density.

Another embodiment of the invention is an isolated DNA molecule useful for the diagnosis or therapy of osteoporosis having sequence homology to a region of the sequence shown in FIG. 2 that does not encode a polypeptide sequence but which is involved in regulation of bone syntheis. Yet another embodiment is a DNA molecule capable of specific hybridization under stringent conditions to a DNA molecule having sequence homology to a region of the sequence shown in FIG. 2 that does not encode a polypeptide sequence, and which, when introduced into human osteoblasts, increases the ability of the osteoblasts to synthesize bone. Yet another embodiment is a DNA molecule at least 15 nucleotides long that is complementary or homologous to a non-protein coding region of the sequence shown in FIG. 2, and which, when introduced into human osteoblasts, increases the ability of the osteoblasts to synthesize bone. In yet another embodiment, the introduced complementary or homologous DNA molecule is at least 25 nucleotides long and in yet another embodiment the DNA molecule is at least 50 nucleotides long. In yet another embodiment, the isolated DNA or RNA molecule that is complementary or homologous to the non-protein coding region of the sequence shown in FIG. 2 is used as a predictor of osteoporosis. In another embodiment the DNA or RNA is used in a binding reaction with DNA or RNA obtained directly or indirectly from a patient sample to to determine propensity of the patient to osteoporosis.

Another embodiment of the invention is a method of using an expression vector to select a test ligand that modulates bone density, comprising the steps of: introducing the expression vector into a host cell to produce a recombinant host cell that expresses the receptor on its surface; culturing the recombinant host cell; and assaying binding between the expressed receptor from the cultured recombinant host cell and the test ligand, wherein the expression vector comprises a promoter that is operably linked with a DNA molecule useful for the diagnosis or therapy of osteoporosis, the DNA molecule selected from the group consisting of: a DNA molecule having a nucleotide sequence encoding an amino acid sequence as shown in FIG. 2; a DNA molecule capable of specific hybridization under stringent conditions to a DNA molecule as described immediately above, and which encodes a protein that, when introduced into human osteoblasts, increases the ability of the osteoblasts to synthesize bone; a DNA molecule having a nucleotide sequence which is degenerate as a result of the genetic code to the encoded protein amino acid sequence accordingly and which encodes a protein that, when introduced into osteoblast cells of humans, increases the ability of the cells to synthesize bone; a DNA molecule capable of specific hybridization under stringent conditions to a DNA molecule according to the first type described above, and which encodes a protein that, when introduced into epithelial cells of humans, increases the ability of the epithelial cells to form vascular tissue; and a DNA molecule having a nucleotide sequence which is degenerate as a result of the genetic code to the encoded protein amino acid sequence accordingly, and which DNA molecule encodes a protein that, when introduced into epithelial cells of humans, increases the ability of the epithelial cells to form vascular tissue.

Yet another embodiment of the invention is a method for discovering a pharmaceutical useful for regulating bone strength or mineralization, comprising the steps: providing a protein reagent comprising at least one ligand binding site of the BMSR protein; contacting the reagent from step (a) with a test substance; and detecting binding between the reagent from step (a) and the test substance, wherein binding indicates that the test substance affects a mineralization reaction upon administration to a patient.

Yet another embodiment of the invention is a method for determining bone strength and mineralization predisposition of a patient from analysis of epitopes on the bone strength and mineralization regulator protein, the method comprising the steps of: providing a tissue or blood sample of the patient; contacting the sample from the first step with at least one conjugate of an antibody or antibody fragment with a reporter molecule, wherein the antibody or antibody fragment recognizes one or more epitopes of the bone strength and mineralization regulator protein; and detecting the formation of a complex between the conjugate and protein within the sample.

Further embodiments will be appreciated from a reading of the contents herein.

### BRIEF DESCRIPTION OF THE DRAWINGS

FIG. 1 shows a list of genes within the six centiMorgan interval on chromosome 11q13 that encode proteins that directly or indirectly affect bone metabolism.

FIG. 2 shows a representative nucleotide sequence (SEQ ID NO:1) of cDNA that codes for human bone strength and mineralization regulation (“BSMR”) protein.

FIG. 3 shows a human bone strength and mineralization regulation (“BSMR”) protein (SEQ ID NO:2) coded for by the DNA of FIG. 2.

FIG. 4 shows nucleotide sequence variation discovered in the coding sequence of the BSMR gene.

FIG. 5 shows representative primers (SEQ ID NOS:3-74) suitable for amplifying cDNA and gDNA molecules useful for detecting polymorphic BSMR genes in humans.

FIG. 6 provides information about constructs that were made according to the procedures shown in the Examples.

FIG. 7 shows the transcriptional regulation of frizzled receptors 1 and 2.

FIG. 8 shows the regulation of elements of the BSMR regulatory system in bone forming cells.

FIG. 9 shows the transcriptional regulation of frizzled receptors and SFRP2 in pluripotent or osteoblast-like cell lines.

FIG. 10 shows the effects of pcDNA3, Wnt3a, Wnt5a, βcatenin and altered βcatenin * on ALP activity.

FIG. 11 shows the effects of various vectors that express representative BSMR effectors on bone metabolism.

FIG. 12 shows the effect of Wnt3a on bone metabolism by ST—2 cells that express different variants of BSMR protein.

FIG. 13 lists some peptides (SEQ ID NOS:75-82) that can act as effectors of BSMR.

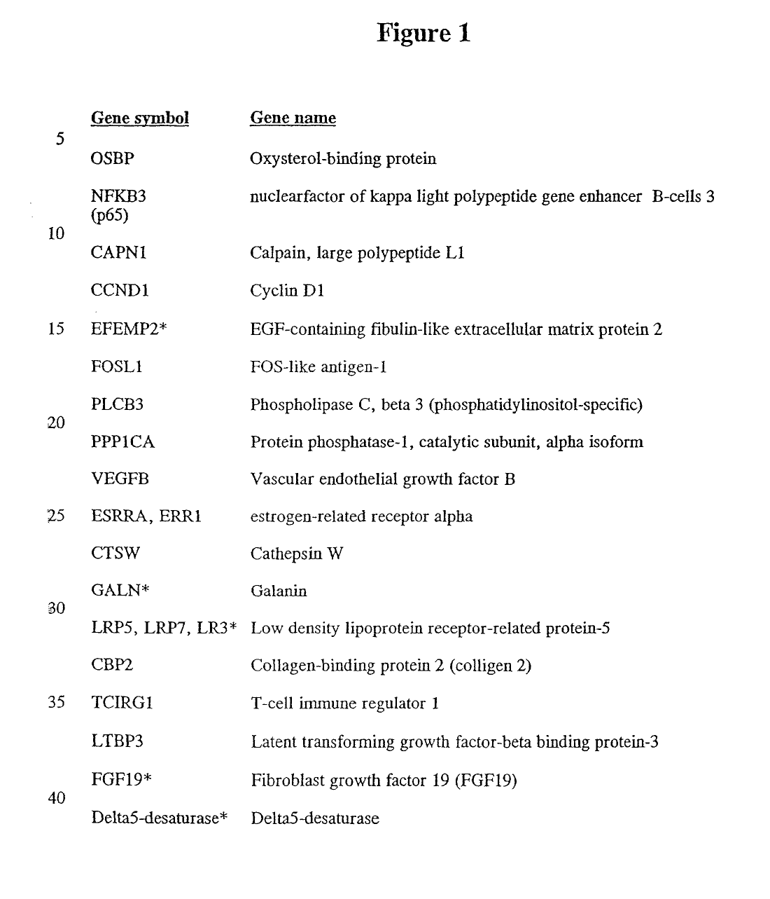

### DETAILED DESCRIPTION OF THE PREFERRED EMBODIMENTS

The inventors have discovered that a mutation in a gene previously known as LRP5, Lrp7, or Lr3 causes OPS and generally affects the regulation of bone strength and mineralization through various molecular interactions. A second discovery is that amino acid alterations at particular locations in that gene, herein termed the “bone strength and mineralization regulator” (“BSMR”) gene lead to genetic polymorphisms that account for a significant portion of normal variability in bone strength and mineralization among individuals. A third discovery is that the BSMR gene modulates biochemical functions leading to alterations in bone strength and density according to its copy number, affinity of binding sites on the BSMR protein (extracellular region adjacent to the membrance surface) to ligand and other biochemical events such as modulating angiogenesis.

A fourth discovery is that certain extracellular ligands can modulate the activity of this protein, and can be applied as therapeutic agents for improving bone strength and mineralization. A fifth discovery is that certain extracellular ligands can modulate the activity of this protein, and can be applied as therapeutic agents for improving bone mineralization. A sixth discovery is that intracellular molecules such as axin, dishevelled, APC, beta—catenin, GSK—3, casein kinase I, casein kinase II, TCF1, TCF3, TCF4, LEF1, groucho, smad and PAR1 should interact with the BSMR gene product and cause changes to biochemical reactions leading to alterations in bone strength and mineralization such as those described by Wehrli et al. in Nature 47: 527 530 (2000); Mao et al. Molecular Cell 7: 801-809 (2001); Wehrli et al. in Nature 407, 527-530 (2000) and Tamai et al. in Nature 407, 530-535 (2000).

These discoveries have led to specific embodiments of the invention that are useful for the diagnosis and treatment (both prophylatic and therapeutic) of osteoporosis. The inventors furthermore have discovered that the BSMR gene system regulates other biological systems such as blood vessel growth in the eye. Although the present disclosure emphasizes diagnosis and treatment of osteoporosis through BSMR regulation of bone cells, other analogous regulatory systems that utilize the BSMR gene are detected and altered in a like manner to that described for bone.

The inventors discovered that an alteration in a single gene (BSMR) can cause OPS and that heterozygous carriers of OPS have low bone densities with respect to the general population. Upon further investigation, the inventors learned that the BSMR gene encodes a transmembrane protein (“BSMR protein”) that regulates bone strength and mineral density. The BSMR protein operates by binding ligands outside the cell and by interacting with intracellular mechanisms. The discovery of the BSMR protein and of the relationship between biochemical parameters of this protein and bone strength and mineralization provides new tools, diagnostics, prophylactics and therapies concerning bone status as well as other systems such as formation of blood vessels in the eye and angiogenesis generally.

More specifically the DNAs, protein, constructs, and methods that employ these materials specifically can increase or decrease bone strength and mineral density due to the discovery that their presence (intact functioning gene product) has a causative relationship with this biological effect. This effect occurs in a non-recessive manner, which proves that the amount of gene product (and/or ligand that binds the gene product) plays an important role in using the DNAs, protein, constructs and methods for detecting and altering this biological phenomenon. Furthermore, some uses inure to the extracellular portion of the protein itself (and/or DNA encoding same). It is known that BSMR is a transmembrane protein having an extracellular part that binds an effector, and an interior part, that engages intracellular machinery to effect a response to bone strength and mineralization upon ligand binding. Accordingly, a specific use of the protein (and or DNA/construct encoding it) is in discovering natural or artificial ligands that bind the extracellular region and which modulate, (particularly up-regulate) bone strength and mineralization. Such tools are very useful because ligand binding modifies an important biological phenomenon that has an important medical consequence.

The known ligand binding regions alone are particularly useful because binding with these regions is linked to a downstream effect such as, in this case, reactions leading to bone strength and mineralization. In other words protein fragments that possess at least one ligand binding site, and preferably three binding sites, (and DNAs that encode them) are very useful in themselves, for detecting ligands that affect bone strength and mineralization. These uses do not depend on the presence of other parts of the protein such as for example, a membrane spanning region or intracellular region because it is known that ligand binding to these sites is an independent reaction that does not necessarily require the rest of the protein.

Accordingly, in a particularly desirable embodiment a BSMR signaling system is provided and used as a “readout” or means to detect a pharmaceutical effector for increasing bone mass. Use of such a “BSMR signaling system” provides a superior screening system over that based on binding to BSMR or a BSMR binding site alone. This is because while a large number of compounds may bind to BSMR a smaller number are capable of sufficiently triggering BSMR signaling that will lead to increased bone mass. This embodiment of the invention thus exploits the new discovery of how the BSMR protein works within an overall regulatory system and provides tools that utilize the overall system, or at least several components of that system working together to obtain more accurate information from screening tests.

In one related embodiment of the invention BSMR is expressed in cells that are competent to travel down the osteoblastic lineage and in which one or more bone metabolic events are triggered thereby. The cell lines used in the examples described herein, are preferred because they contain the BSMR anabolic machinery suited for control of bone growth by a BSMR effector. Other cell lines that can become committed to bone formation or that are already committed are particularly useful in embodiments of the invention for this reason.

Alkaline phosphatase activity is exemplified as a preferred metabolic reaction in many embodiments. However, a skilled artisan is also familiar with a number of other chemical and/or physical reactions that indicate bone anabolism and which can be used. One preferred technique is to monitor the binding of BSMR with another effector by using fluroescence resonance energy transfer between a fluor on BSMR and a fluor on the effector as the two fluors become close through binding of effector to BSMR. This technique works well to monitor binding of BSMR with axin and other components of the BSMR regulatory system as well. Accordingly one embodiment is to use this technique in pluripotent cell lines that can go down the osteoblastic lineage or even in osteoblastic cell lines to find extracellular ligands (BSMR effectors) for BSMR signaling.

Cell lines were created that stably express fluorescently-tagged BSMR and fluorescently-tagged axin. These cell lines were capable of differentiating into osteoblastic cells in the presence of BMP2. Thus, the cells either already express, or are capable of expressing other cell surface molecules, cytoplasmic proteins, and nuclear proteins that are required for osteoblastic differentiation. A skilled artisan will appreciate other cell lines that share these characteristics and that can be used as well. Ligands that can lead to fluorescence resonance energy transfer between components of the BSMR regulatory system, in these stable cell lines, can have greater specificity for effecting normal BSMR mediated osteoblastic proliferation and/or differentiation.

The use of a specific bone anabolism reaction (or conversion of a cell into a developmental pathway) in combination with the BSMR system is greatly preferred for screening of pharmaceutical candidates as it represents an improvement in the sensitivity and/selectivity of the screening technique.

An embodiment of the invention provides a ligand discovery system that identifies pharmaceutically active substances that can bind BSMR and affect bone strength and mineralization. The inventors have discovered that upregulating this bone strength and mineralization system can improve bone status not only in individuals having a defective gene, but also in normal individuals that may desire increased bone strength or mass. This embodiment of the invention is carried out by one or more of: (a) adding an exogenous pharmaceutical that binds to BSMR; (b) altering the concentration of a naturally occuring ligand that binds to BSMR; (c) activating a “pathway gene” that encodes a product that is associated with the BSMR protein regulatory pathway; (d) transgenically adding the BSMR gene or a pathway gene to desired cells; (e) activation of a BSMR gene in bone cells; (f) inactivating a pathway gene that encodes a product that is associated with the BSMR protein regulatory pathway and (g) adding an exogenous pharmaceutical that affects the BSMR protein regulatory pathway.

In preferred embodiments a ligand is added either by application as a pharmaceutical or by transgenic expression by an introduced gene that encodes the ligand. The applied or introduced ligand acts upon the BSMR protein to increase bone strength and mineralization. Examples of ligands contemplated for this embodiment of the invention include the human Wnt genes, other related cysteine rich genes such as the 36 kDa frizzled related protein, dkk—2, dkk—1, dkk—3, Frzb—1, apolipoproteins that are potential ligands for LRP5, other related cysteine rich proteins such as the CCN family, which includes WISP1-3, CTGF, NOV, and Cyr61 among its members.

In other embodiments biological functions that are controlled by the BSMR gene similarly may be regulated by any of (a) to (e). In yet other embodiments, the BSMR gene, or a portion that codes for the ligand binding portion of the BSMR protein is combined with another characteristic sequence that is useful to monitor the protein or to immobilize it. Example 2 discloses use of a FLAG antibody epitope in this context, and Example 3 demonstrates insertion of a human IgG1 Fc domain, which can detect BSMR polypeptide, as well as having the BSMR engage in binding reactions (or immobilization to a solid surface) based on reactivity of the added Fc domain with an Fc binding partner, as is known in the art.

In another embodiment a portion of the BSMR protein containing one or more ligand binding regions is used. The examples demonstrate the use of a BSMRdelta TM decoy construct. The construct has carboxyl-terminal myc and HIS tagged epitopes that are fused in frame with the BSMR extracellular domain, which contains the ligand binding sites. This fusion protein was found to be readily secreted from cells and functional as an inhibitor of Wnt signaling in vitro. Still further, the fusion protein was found to inhibit bone accrual in calvarial explant cultures. This protein and others like it that a skilled artisan may derive based on this disclosure is particularly useful for drug design, discovery of related biochemistry relating to the Wnt system and as a therapeutic itself. Such a fusion protein can be purified, for example, using a nickel column or an anti-mycantibody column.

Preferable constructs made to demonstrate this embodiment are BSMRdeltaC, BSMRdeltaTM and BSMR—YFP. BSMR—YFP is particularly useful to determine ligand driven interactions between BSMR and a cytoplasmic interacting protein such as axin as may be carried out, for example using fluorescence resonance energy transfer (“FRET”).

Many of the preferred embodiments relate to fundamental aspects of cellular regulation that have many repercussions in an organism. Hence, embodiments of the invention pertain more broadly to a new transmembrane protein based regulatory system that affects other systems as well. Furthermore, the BSMR gene may be used as a tool in genetic engineering, particularly in combination with other genes involved in the BMSR regulatory sequence.

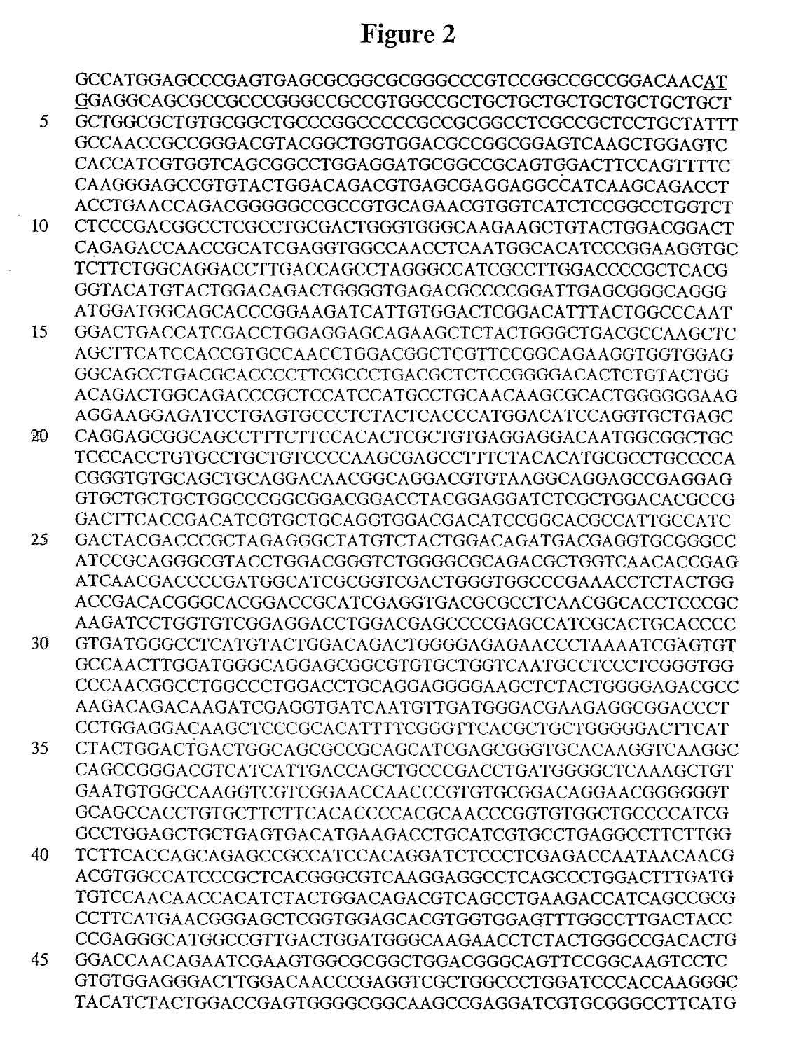

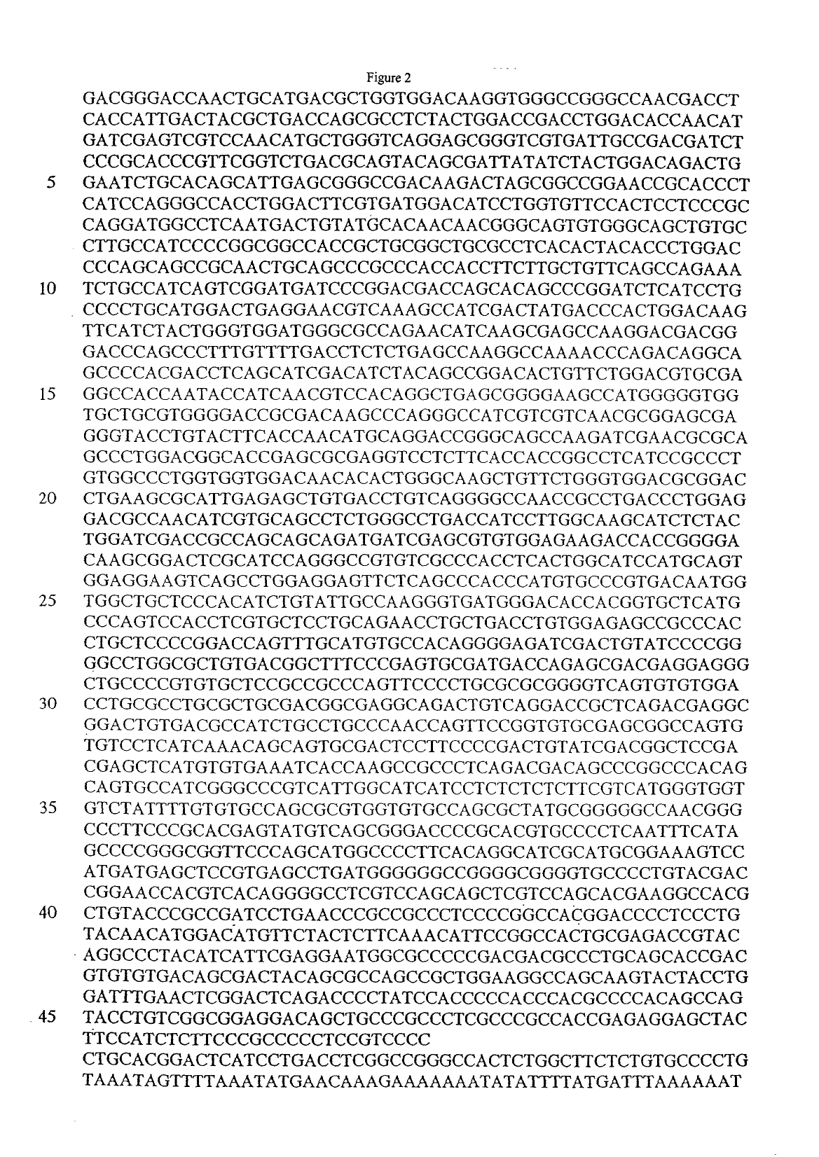

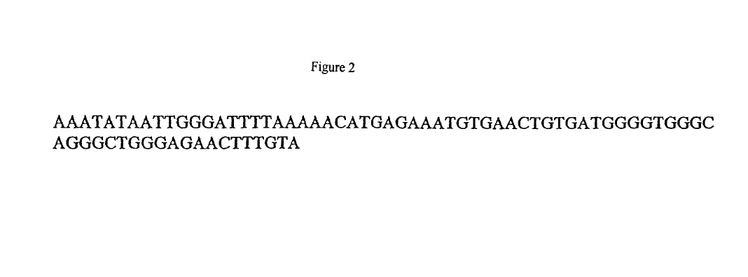

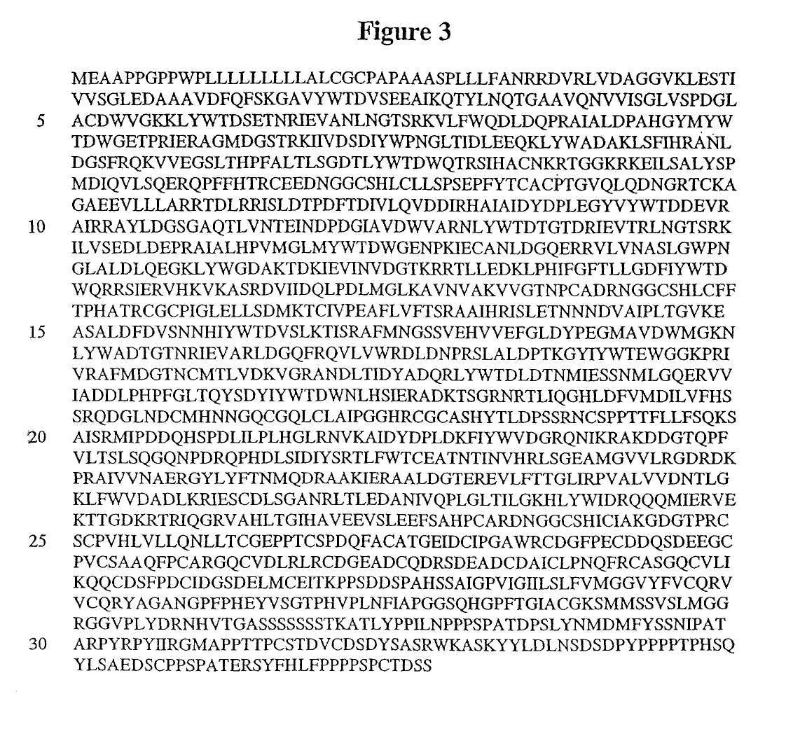

Many embodiments of the invention relate to the creation and use of DNA, RNA, peptide and/or protein comprising specific sequences. Preferred sequences for many embodiments are shown in the Figures. FIG. 2 depicts the cDNA sequence of a BSMR messenger RNA. The translation start codon “ATG” begins at residue 51 and is underlined. FIG. 3 shows an entire amino acid sequence of a protein corresponding to the nucleic acid coding sequence of FIG. 2. And another embodiment the non-transcribed regulatory sequences upstream of the coding sequences are detected and used to monitor bone status. Those sequences are known to the skilled artisan and can be found in the gene called LRP5. Allelic variants in the regulatory region, namely the promoter and inhancer are particularly useful for determining bone status. Of course, inhancer region(s) effecting bone status also may be found downstream of the gene as well. Accordingly, one use of the invention is to take a sample of biological material from a patient and look for a change to a regulatory sequence, which relates to bone status.

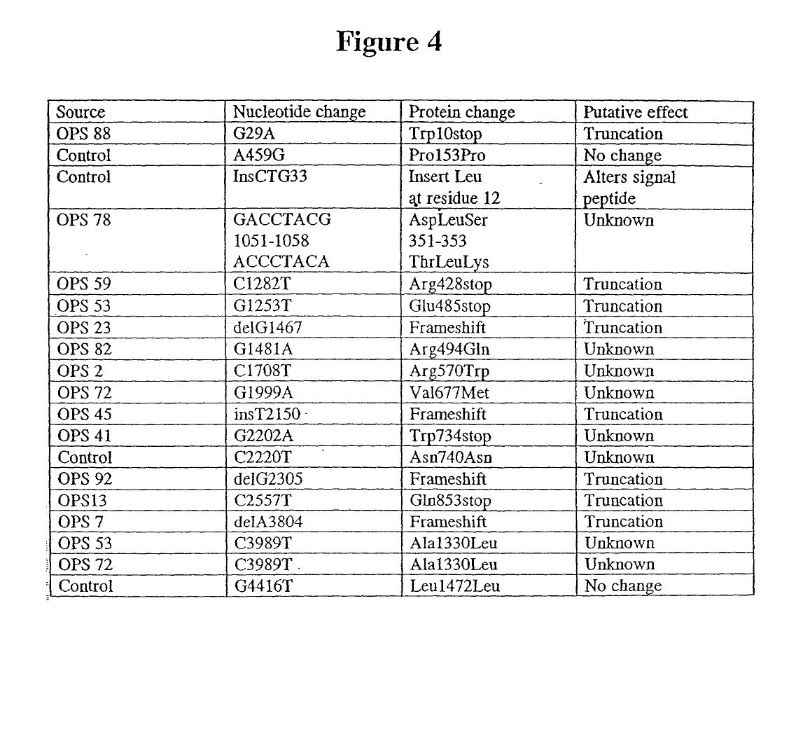

FIG. 4 shows some BSMR coding sequence variations that have been found by the inventors. The term “Source” in this figure refers to the genomic DNA source. The term “OPS” indicates that the respective DNA came from affected OPS patients. The term “Control” indicates that the DNA studied was anonymous DNA samples from a healthy individual. The inventors have discovered the third listed alteration of a leucine insertion into residue 12, within a leucine stretch within the signal peptide region of the protein at a frequency of approximately 20 of 100 control individuals. This yields an estimated allele frequency of 0.1. One embodiment of the invention is a diagnostic tool for detecting the addition of one, two, three, four, five or more leucines to this stretch of leucines, as addition of one or more leucines here has an effect on the protein's functioning. The detection of this polymorphism and related leucine addition polymorphisms within this particular polyleucine spot is pronostic for osteoporosis tendency and normal bone density tendency. Detection of such leucine insertion polymorphisms specifically is contemplated for embodiments of the invention. In one embodiment multiple leucines are added to the region.

In another embodiment, addition of one, and especially addition of more than one leucine decreases the chances for removal of the signal peptide region, allowing the polyleucine spot to remain on the protein during processing and transport to the cell membrane and become exposed at the outer cell surface. Thus, this polyleucine expression modulates the protein's effects and affects bone density. In this context, the inventors note that duplications of 18 bases and 27 bases may occur leading to insertion of 6 and 9 leucines, respectively in the signal peptide region of the receptor activator protein TNFRSF11A and that similar expansion of leucines within the protein of the present invention can occur. See, for example, Nat. Genet. 24(1): 45-48 (2000). Detection of such expansion (that is addition of 1, 2, 3, 4, 5, 6, 7, 8, 9, 10 or more leucines) allows detection of the proteins's affected activity, in particular an increase in activation of the regulatory activity of this protein and is contemplated in an embodiment of the invention.

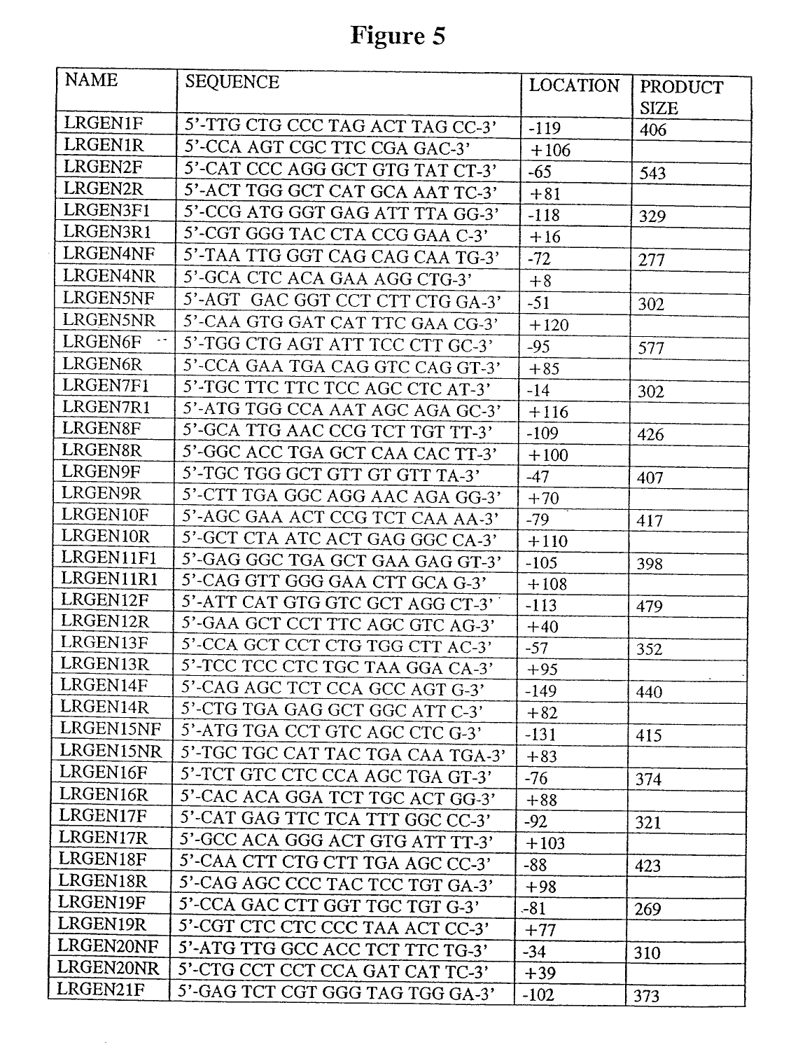

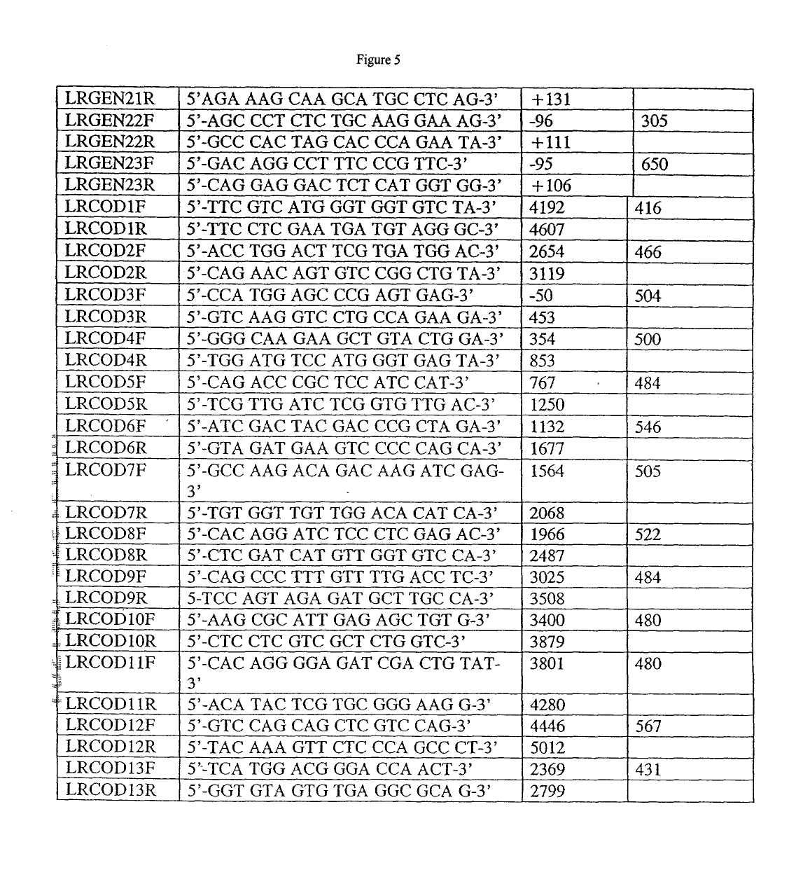

FIG. 5 presents representive oligonucleotide PCR primer sequences that have been used to amplify BSMR sequences from genomic DNA. These data identify osteoporosis associated genetic variants. The term “Location” in this figure refers to the position of the 5′nucleotide relative to the intron/exon boundary. The term “Product size” refers to the length of the PCR amplimer in basepair units. Nucleic acid and protein that embody one or more of these sequences specifically are contemplated for the invention.

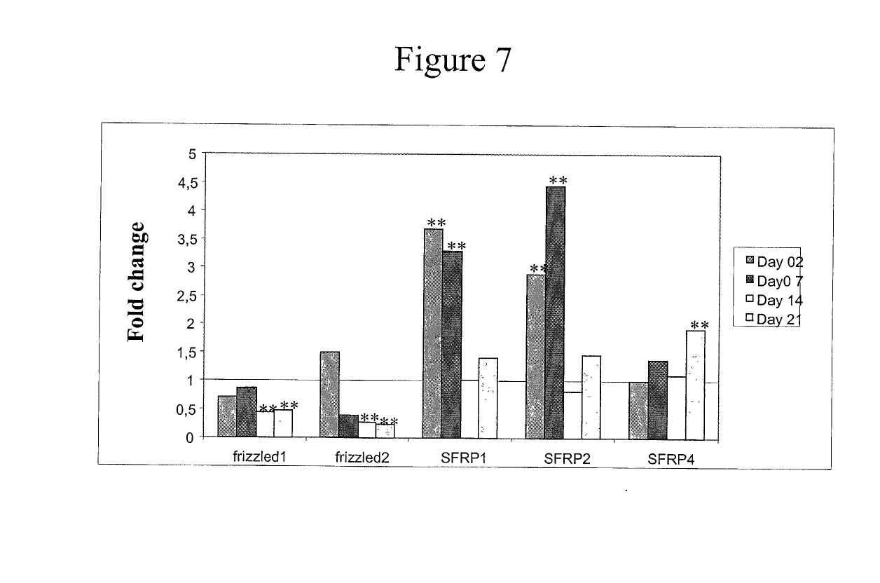

FIGS. 7 to 12 demonstrate results showing use of BSMR effector molecules such as Wnt proteins in upregulating the BSMR system, leading to increased metabolism related to bone strength and mineralization.

### Definitions

To facilitate review of the embodiments and to provide greater clarity to the claims the following definitions for often-used terms are provided, which are consistent with usage in the field.

“BSMR gene” is a gene described herein, the extreme mutant forms of which are associated with the disease OPS. This definition is understood to include the various sequence polymorphisms that exist, wherein nucleotide substitutions in the gene sequence may affect the functioning of the gene product. This term relates primarily to an isolated coding sequence, but also can include some or all of the flanking regulatory elements and/or intron sequences. The mouse homologue of this gene is referred to as the murine BSMR gene.

“BSMR protein” is a protein encoded by the human BSMR cDNA. This definition is understood to include the various sequence polymorphisms that exist, wherein amino acid substitutions in the protein sequence do not completely abolish the functions of the protein.

“cDNA” refers to complementary DNA, which lacks internal, non-coding segments (introns) and lacks regulatory sequences which determine transcription. cDNA is synthesized in the laboratory by reverse transcription from messenger RNA extracted from cells.

“DNA” refers to deoxyribonucleic acid. DNA is a long chain polymer which comprises the genetic material of most living organisms (some viruses have genes comprising ribonucleic acid). The repeating units in DNA polymers are four different nucleotides, each of which comprises one of the four bases, adenine, guanine, cytosine and thymine bound to a deoxyribose sugar to which a phosphate group is attached. Triplets of nucleotides, referred to as codons, in DNA molecules code for amino acids that are linked in a polypeptide. The term codon also is used for the corresponding (and complementary) sequences of three nucleotides in the mRNA into which the DNA sequence is transcribed. A standard nomenclature for DNA bases is employed.

“BSMR Effector” refers to a molecule that has an effect on the BSMR signaling pathway, and includes without limitation, BSMR agonists, BSMR antagonists, intracellular factors that interact with BSMR, intrinsic membrane proteins that interact with BSMR, extrinsic membrane proteins that interact with BSMR and pharmaceuticals that are found to interact with any of the parts of the BMSR signaling pathway.

“Hybridizations” preferably are performed under stringent conditions, for example, at a salt concentration of no more than IM and a temperature of at least 25 degrees C. For example, a condition of 5X SSPE (750 mM NaCl, 50 mM NaPhosphate, 5 mM EDTA, pH 7.4) and a temperature of 25-30 degrees C. is suitable for allele-specific probe hybridizations.

“Hybridization probes” are oligonucleotides capable of binding in a base-specific manner to a complementary strand of nucleic acid. Such probes include peptide nucleic acids, as described in Nielsen et al., Science 254, 1497-1500 (1991).

“Isolated” means that that the material has been removed from its original environment. For example, a naturally occurring DNA molecule present in a living animal is not isolated, but the same DNA molecule, separated from some or all of the coexisting materials in the natural system, is isolated.

An “isolated nucleic acid” refers to the predominant species present. That is, on a molar basis the isolated nucleic acid it is more abundant than any other individual species in the composition. Preferably, an isolated nucleic acid comprises at least about 50, 80 or 90 percent (on a molar basis) of all macromolecular species present. Most preferably, the object species is purified to “essential homogeneity” namely, that contaminant species cannot be detected in the composition by conventional detection methods.

“Linkage” describes the tendency of genes, alleles, loci or genetic markers to be inherited together as a result of their location on the same chromosome, and can be measured by percent recombination between the two genes, alleles, loci or genetic markers.

An “oligonucleotide” can be DNA or RNA, and single-or double-stranded. Oligonucleotides can be naturally occurring or synthetic, but are typically prepared by synthetic means. Preferred oligonucleotides of the invention include segments of DNA, or their complements, and may include any one of the polymorphic sites shown in the Figures.

“ORF” means open reading frame, which contains a series of nucleotide triplets (codons) coding for amino acids without any termination codons. These sequences are usually translatable into protein.

A “pathway gene” encodes a product, which may be regulatory RNA or a protein that is associated with the BSMR gene product or with the biochemical pathways that extend to (for example, upstream signals) or from (for example, downstream signals) the BSMR gene product.

“PCR” means polymerase chain reaction. This describes a technique in which cycles of denaturation, annealing with primer, and then extension with DNA polymerase are used to amplify the number of copies of a target DNA sequence.

“Polymorphism” refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. The first identified allelic form may be designated arbitrarily as a reference and other allelic forms designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

“Primer” refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified.

The term “purified” does not necessitate absolute purity but relative purity. A reference to a protein as “purified” means that the protein is more pure with respect to the same protein in its natural environment within a cell or extracellular space from which it was purified.

A “single nucleotide polymorphism” usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms also can arise from a deletion of a nucleotide or an insertion of a nucleotide relative to a reference allele.

Additional definitions of specific terms used in receptor signaling biology are, for example, found in Mao et al., Molecular Cell, Vol. 7, 801-809 (2001) and common terms used in molecular biology may be found in Lewin, B. “Genes IV” published by Oxford University Press.

### Detection of OPS Disease Carriers by DNA Analysis

An important discovery related to many embodiments of the invention is that a single regulatory protein from the BSMR gene influences bone strength and mineralization.

Using known techniques (Gong et al., Am. J. Hum. Genet. 59: 146-151 (1996) the inventors extensively studied DNA of individuals afflicted with OPS. During those studies five severely afflicted patients were found who had consanguineous parents (parents having the same parents) and were expected to have inherited two copies of the same genes that might affect OPS. All five patients and two other severely afflicted patients were found to have frame-shift or nonsense codon mutations in a single gene, variously named the “LRP5,” “Lrp7” and “Lr3” gene, as described in Example 1.

The linking of mutation results as seen in Table 1 with OPS symptoms indicates that a defect in a single gene (LRP5, Lrp7, Lr3a ) causes this disease. Accordingly, one embodiment of the invention is a method for detecting homozygous or heterozygous carriers of OPS disease by DNA analysis. In a preferred embodiment a DNA sample is taken from a subject and a major alteration in the BSMR gene is assayed for. The term “major alteration” in this context means that the gene has a frame shift, termination mutation, or other alteration that causes a loss of function. This loss could be caused by a frame shift or termination mutation that truncates the protein or by a missense mutation that interferes with protein folding, ligand binding, or downstream signaling.

The insight from linking OPS disease with a major alteration in the BSMR gene, combined with further observations that OPS mutations are not true recessives led to the understanding that polymorphisms in this gene could account for much of the phenotypic variation in bone density among “normal” individuals. Since carriers of OPS mutations have reduced bone mineralization density compared to the general population, other types of genetic variation within BSMR also will affect bone mineralization density. Still further, the inventors have explored biochemical characteristics of this regulatory protein and made further discoveries that potentiate new compositions and methods for the detection of and improvement of bone status. These discoveries provide various nucleic acid technologies for diagnosis and intervention of bone strength and mineralization, as further described below.

The BSMR Gene Codes for a Transmembrane Protein that Regulates Bone strength and Mineralization

The inventors discovered that the regulatory protein responsible for bone density status is a transmembrane protein having an intracellular portion that has been shown to lack a conserved NPXY motif for coated pit-mediated endocytosis. The inventors have linked that cytoplasmic portion sequence with a function, namely the downstream regulation of bone strength via binding of one or more regulatory ligands to the one or more binding sites on that portion of the BSMR protein. In one embodiment according to the invention a pharmaceutical compound affects bone strength and mineralization by interacting with or replacing the function of the intracellular portion of the protein.

The extracellular portion of the native BSMR protein is thought to contain three ligand binding repeats adjacent to the membrane attachment point. According to an embodiment of the invention one or more of these three ligand binding sites is modified and increases the BSMR protein's effect on bone strength. In another embodiment a gene that encodes the modified protein is introduced into cells by a viral vector. In yet another embodiment of the invention a protein or polypeptide that contains at least one of these ligand binding sites is used in a binding assay to find a pharmaceutical compound that has an effect on bone strength and mineralization.

The BSMR protein has biochemical features that allow the protein to regulate bone density. One feature is the presence of three “LDLR repeat” ligand binding segments adjacent to a transmembrane region that anchors the mineralization ligand binding portion of the protein exterior to the membrane but close to the membrane surface. Without wishing to be bound by any one particular theory of their invention, the inventors believe that BSMR binds ligand and operates by stimulating the proliferation of bone cell progenitors, their further differentiation into bone forming cells (osteoblasts), or the enhanced synthesis of bone by fully differentiated cells. In response to binding ligand the BSMR protein mediates these effects through protein interactions that occur on the cytoplasmic side of this transmembrane protein.

In one embodiment a regulatory protein useful for practice of the invention has a protein sequence described in FIG. No. 2 or a variant of such sequence. Through the manipulation of a nucleotide sequence by standard molecular biology techniques, variants of the BSMR protein may be made which differ in precise amino acid sequence from the disclosed proteins yet which maintain the essential characteristics of the BSMR protein or which may be selected to differ in some characteristic way. Such variants are another embodiment of the invention.

A Wide Variety of BSMR Effectors Influence Bone Mineralization

Any molecule, either naturally occurring or synthetic which reacts with one or more components of the BSMR pathway (typically by binding to a member of that pathway) is a BSMR effector and is useful for altering bone status in patients. In addition, these effectors can affect angiogenesis, as demonstrated in Example 13.

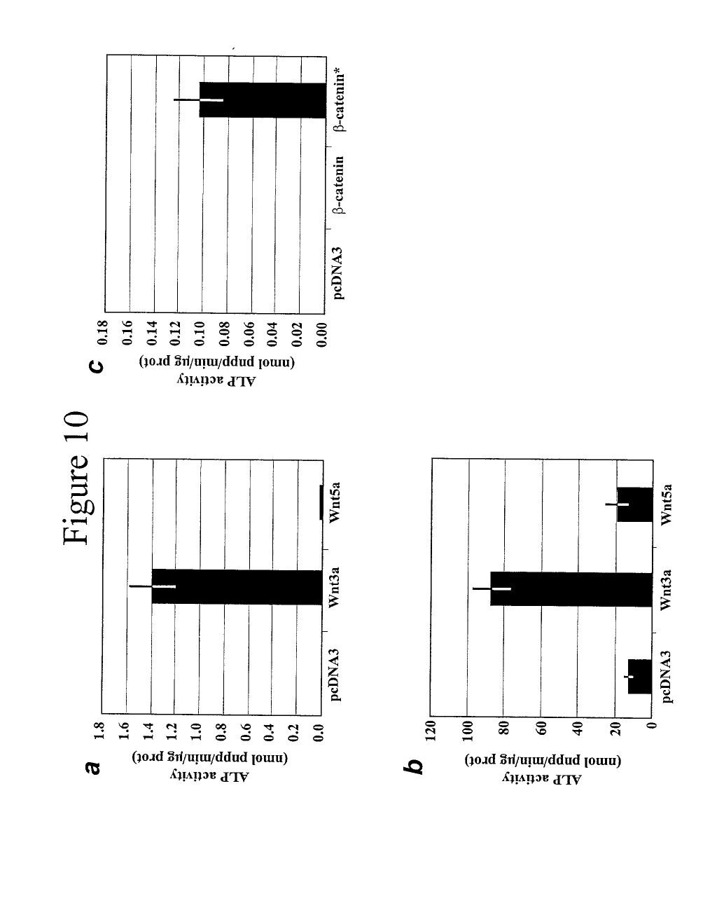

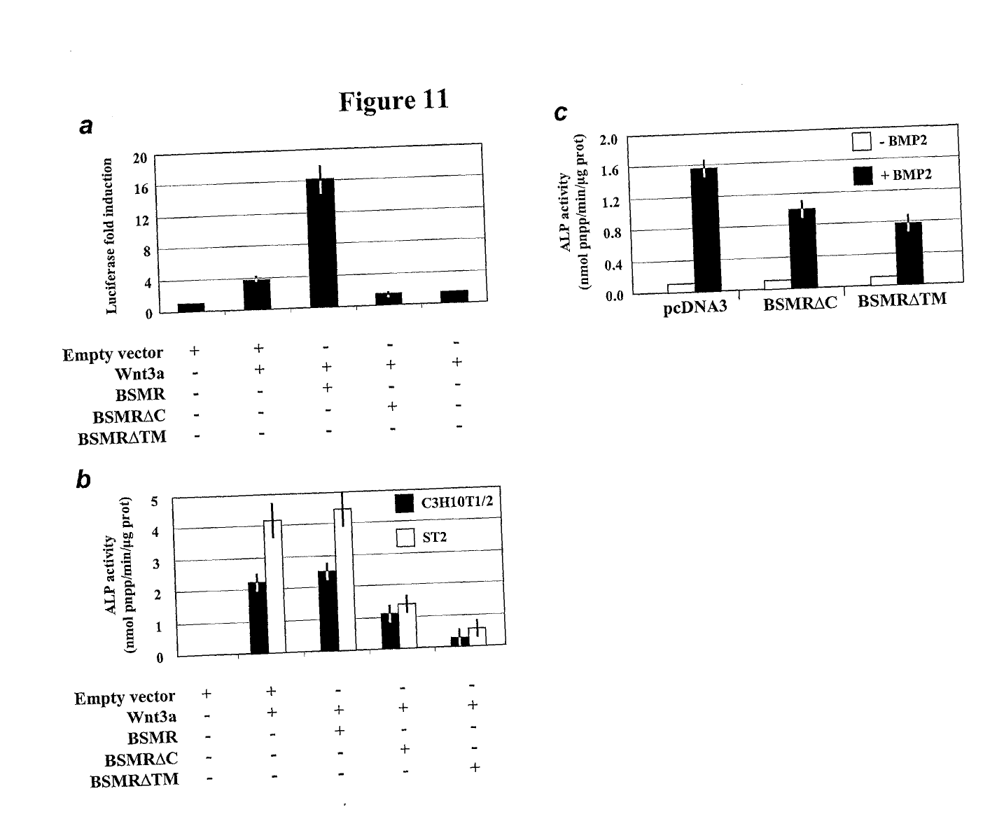

Particularly advantageous embodiments of the invention utilize as BSMR effectors, extracellular molecules such as Wnt proteins and intracellular factors such as axin, dishevelled, APC, beta—catenin, GSK—3, casein kinase I, casein kinase II, TCF1, TCF3, TCF4, LEF1, groucho, smad and PAR1) involved in the Wnt/β—catenin signaling pathway, to alter bone status and other biological features such as angiogenesis. Exemplary studies were carried out to demonstrate these embodiments, as shown in Examples 4 to 13. Some of the studies used pluripotent mesenchymal cells that were induced into the osteoblastic lineage by exogenous growth factors. FIGS. 10 to 12 show data obtained from use of effectors from the Wnt/β—catenin signaling pathway acting upon these cells in a Smad independent manner. Other studies further demonstrated that BSMR generally is involved in this pathway, since reducing LRP5 function with either a transmembrane anchored dominant negative receptor or a secreted decoy receptor interfered with Wnt-induced alkaline phosphatase stimulation, as shown by the data of FIG. 11. Example 13 reports data showing that effectors such as, for example, proteins that bind ligand and down regulate the BSMR system are useful inhibitors of angiogenesis and have a role in, for example, combating tumor growth. These effectors, and in particular, negative regulators of BSMR are particularly contemplated as anti-cancer pharmaceutical agents.

In another embodiment of the invention an effector that contains at least one BSMR binding site decreases bone mineralization. Data that demonstrates this embodiment are shown in FIG. 11. This figure indicates that adding BSMR ligand binding sites (a secreted decoy receptor) decreases the expression of alkaline phosphatase and bone formation in mouse primary osteoblastic cell cultures and calvarial explant cultures, respectively. These data also indicate that a molecule that binds BSMR such as an anti-BSMR antibody fragment, whole antibody or other specific BSMR binding molecule if present can increase alkaline phosphatase (a metabolic reaction or intracellular mechanism indicative of bone mineralization) leading to bone formation. The term “specific BSMR binding molecule” means a molecule that binds to BSMR with an affinity constant that is at least 10 fold higher, preferably 100 fold higher and more preferably 1000 higher than the affinity constant for the molecule's binding to human serum albumin.

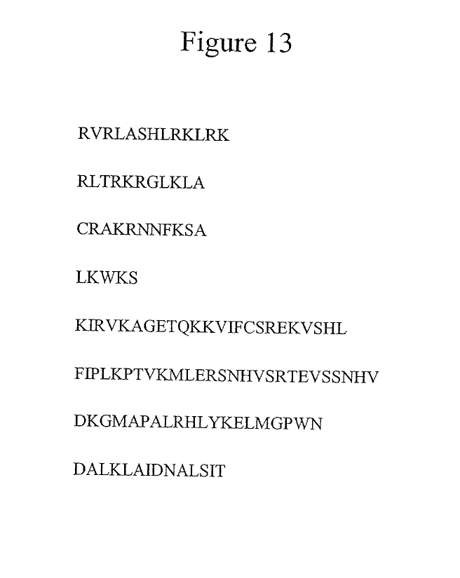

Of course, peptides related to the above described proteins will have activity in influencing the BSMR regulatory system and are desireable for embodiments of the invention. The peptides shown in FIG. 13 in this context are desirable and generally may stimulate the BSMR system, leading to increased bone metabolism, as could be seen by measuring alkaline phosphatase activity or another metabolic reaction affecting bone mineralization.

Other gene products have roles in bone biology and are useful for altering bone mineralization according to embodiments of the invention. In particular embodiments of the invention an effector of BSMR such as one listed herein is administered together with one of these other gene products. In particular, genes and their encoded products that are targets of Wnt/β—catenin signaling are particularly useful in this context. By “administered together with” is meant that the BSMR effector is introduced into the body close enough in time to effect the BSMR system synergistically with the other product. For example, BMP2 may be added by injection one or more times over a three day period to achieve a sufficient level after which a BSMR agonist may be introduced orally or by injection. The BSMR acts upon cells that contain higher BSMR levels due to the earlier BMP2 administration.

Among agents particularly suitable for coadministration are BMP2, fibronectin, which is necessary for osteoblast development and survival, fra-1, a transcription factor that enhances bone formation, the osteoblast-specific factor, and periostin. Of course, for many embodiments combinations of these may be used to create a synergistic effect in improving bone strength, as determined by increased ALP activity.

Extracellular Effectors of the BSMR Transmembrane Protein for Therapy and Prophylaxis

In many embodiments extracellular ligands are particularly desirable to modulate bone strength and mineralization. The ligands operate by binding to one or more extracellular ligand binding repeats of the regulatory protein and thereby activate biochemical and cellular events that lead to increased bone strength and mineralization. In one embodiment, the ligand achieves this effect by causing a cell which expresses the regulatory protein on its surface, to proliferate and/or differentiate into an osteoblast cell. In another embodiment the ligand achieves this effect by inhibiting a cell which expresses the regulatory protein on its surface from proliferating or from differentiating into an osteoclast cell. In yet another embodiment the ligand achieves this effect by by removing a catabolic extracellular matrix factor such as an enzyme or growth receptor by binding to the matrix factor and preventing the factor from interacting with other substances.

In a preferred embodiment the extracellular ligand causes improved bone strength and mineralization by binding to the extracellular region of the BSMR protein. Examples of these ligands are the Wnt proteins, such as the human Wnt proteins such as WNT1 found on chromosome 12q13 and previously known as INT1, WNT2 found on chromosome 7q3.1 and previously called IRP, WNT2B/13 found on chromosome 1P13, WNT3 found on chromosome 17q21, WNT3A found on chromosome 1q42.13 and linked to WNT14, WNT4 found on chromosome 1, WNT5A found on 3p14-p21, WNT5B found on chromosome 12, WNT6 found on 2q35, WNT7A found on 3p25, WNT7B found on 22q13, wnt8a found on chromosome 5, WNT8B found on 10q24, WNT10A found on chromosome 2, WNT10B found on 12q13.1, WNT11 found on 11q3.5, WNT14 found on 1q42, WNT15 found on 17q21, WNT16 found on 7q31; the 36 kDa cysteine rich frizzled related protein, Frzb—1, apolipoproteins that are potential ligands for LRP5, and other related cysteine rich proteins such as the CCN family of CFTG, NOV, wisp3 and Cyr61.

Of the extracellular effectors, the Wnt proteins were found by experimentation to be particularly useful for embodiments of the invention. In one embodiment BSMR mediated bone mineralization is up regulated by a Wnt protein that is introduced into the body directly as a pharmaceutical, or that is introduced indirectly by transgenic expression, or by increasing the amount made by bone forming cells. Without wishing to be bound by any one theory of the invention, it is believed that BSMR functions as a Wnt co-receptor in the canonical signaling pathway that employs β—catenin as a downstream effector. Accordingly Wnt up regulates the BSMR system and the Wnt antagonist Dickkopf down regulates the system by interfering with Wnt signaling by binding to BSMR (LRP5). See Examples 10 to 12, which show the use of Wnt proteins as effectors of the BSMR system in cells that can differentiate along the osteoblastic lineage in vitro. These examples show transient transfection experiments using different Wnts and their effects on induction of expression of the osteoblastic markers alkaline phosphatase (Alpl), osteocalcin (Bglap), and Cbfal, in the pluripotent mesenchymal cells C3H10T1/2 and ST2.

In the embodiment shown in Example 10, Wnt3a acts as an effector of BSMR activity and induces alkaline phosphatase activity in both cell lines studied (See FIG. 10a and 10b), without having a significant effect on osteocalcin or Cbfa1 gene expression (data not shown). This induction was inhibited by co-expression with the negative effector Dickkopf (data not shown). In another embodiment, a BMP inhibitor (Noggin) was increased by over-expression yet this inhibitor did not affect Wnt3a-mediated alkaline phosphatase activity. A less preferred effector is Wnt5a, which does not elevate the level of cytosolic β—catenin, nor activate LEF1-dependent transcriptional activity, and was found not to affect alkaline phosphatase activity in the C3H10T1/2 and ST2 cells studied (FIGS. 10a and 10b). A further example of an effector according to embodiments of the invention is the action of an active mutant form of β—catenin (β—catenin*), which induced alkaline phosphatase activity in C3H10T1/2 and ST2 cells (see FIG. 10c). Each of the components in the Wnt canonical pathway may be used in some respect to alter bone status and each component specifically is contemplated for embodiments of the invention.

Example 11 (see FIG. 11) demonstrates the effect of BSMR overexpression.

FIGS. 11a to 11c show the effect of inducing alkaline phosphatase activity in pluripotent cells that enter the osteoblastic lineage, and the effect of Wnt3 on overexpressed BSMR. FIG. 11a shows enhancement of Wnt3a-mediated TCF—1 activation by overexpresson of BSMR in COS cells. Accordingly, in embodiments of the invention, BSMR is increased, causing the effects demonstrated by these working examples. Methods of increasing expression, for example by transgenic expression of foreign genes, gene regulators and the like are known and new methods will be developed in the future for upregulating BSMR, now that the link between the BSMR gene and bone mineralization is established.

Each of these methods is contemplated as a therapy for improving bone mineralization status of a patient who desires an altered bone mineralization status. One such embodiment is a means for improving bone mineralization status of a patient with demonstrably insufficient bone mineralization, wherein the means is addition of a compound that is known or found to increase expression of BSMR, transgenic expression of a foreign BSMR gene or gene regulatory unit, and introduction of allogenic or autologous cells into a patient, wherein the introduced cells have been treated to increase their expression of BSMR on their surfaces, and wherein the cells are capable of becoming osteoblast cells after administration to the patient.

In another embodiment of the invention, modifications are made to a known or discovered ligand or Wnt protein or protein derivative that maintain or increase the effect of the ligand on bone strength and mineralization. Preferably a modification is determined in a routine matter to have an increased affinity between ligand and BSMR protein. In a preferred embodiment in this context to increase binding, a ligand is dimerized, either by making a peptide into a di-peptide conjugate or by some other covalent reaction linking two ligands together. In yet another embodiment a ligand is trimerized, and in another, higher multiples are used.

In another desirable embodiment an extracellular antagonist alters bone strength and mineralization by interfering with binding of a stimulatory ligand to the BSMR protein. Some examples of stimulatory ligands are given in FIG. 13. For example, dkk—1, dkk—2, dkk—3, RAP, sFRP and human analogues of these proteins can interfere and have various degrees of activity. In a preferred embodiment a polynucleic acid encoding a gene for a naturally occuring antagonist of BSMR activity is added in an antisense format such that an antisense strand forms and binds to messenger RNA, thus blocking translation and/or triggering nuclease destruction of the RNA. Recombination using antisense technology has previously been used to inhibit expression of specific gene products in mammalian cell lines (Kasid et al., Science 243:1354-1356 1989; Khoka et al., Science 243:947-950 1989; Izant et al., Science 229:345-352 1985) including some retroviruses; (von Ruden et al., J. Virol. 63:677-682 1989; and Chang et al., J. Virol. 61:921-924 1987). Each of these methods, and later developed methods specifically are contemplated for this embodiment of the invention.

Intracellular Effectors of the BSMR Transmembrane Protein

BSMR operates in many embodiments through intracellular partners that interact with the carboxyl terminal side of the protein inside the cell. One desirable embodiment is a technique for identifying intracellular partners with a “yeast two-hybrid” or “pull-down method” and which utilizes the cytoplasmic tail of BSMR or another peptide derived from the tail. Experiments were performed that confirmed this strategy. Using the yeast two hybrid system, a set of genes were found that interact with the BSMR protein cytoplasmic tail.

Several intracellular partners of BSMR were identified as members of the putative downstream signaling system for BSMR from study with the yeast-two hybrid system. Each of these putative BSMR effector molecules may be altered, by chemical agents or genetically, to influence bone strength and mineralization through the effects on BSMR. A skilled artisan readily will appreciate alternative ways of altering the levels of each of these proteins and/or affecting their interaction with the BSMR system. In an advantageous embodiment a chemical is used to affect the activity of the respective compound in vivo, indirectly leading to altered bone status.

The putative intracellular partners useful for controlling bone status are: Mus musculus FK506 binding protein 8, as described in Br J Pharmacol 1998 Jul.;124(5):849-56; Mus musculus nuclear protein 95 (Np95) as described by Fugimori et al in Mamm. Genome 9 (12), 1032-1035 (1998); GLI-Kruppel family member GLI3 [Mus musculus] as described by Thien in Biochim. Biophys. Acta 1307 (3), 267-269 (1996); Mus musculus RAN binding protein 9 (Ranbp9) as described by Doi and Watanabe at http://www.ncbi.nlm.nih.gov/entrez/ query.fcgi?cmd=Retrieve&db=Nucleotide&list₁₃ uids=09910285&dopt=GenBank; Mus musculus ISL1 transcription factor, LIM/homeodomain as described by Koehler and Dear at http://www.ncbi.nlm.nih.gov/entrez/query.fcgi?cmd=Retrieve&db=Nucleotide&list₁₃ uids=10946847&dopt=GenBank; Human signal-transducing guanine nucleotide-binding regulatory (G) protein beta subunit as described by Gao et al in Proc. Natl. Acad. Sci. U.S.A. 84, 6122-6125 (1987); Mus musculus, casein kinase II, beta subunit as described by Strausberg at http://www.ncbi.nlm.nih.gov/ entrez/viewer.cgi?val=13277764; Homo sapiens zinc finger protein 198 (ZNF198) as described by Wiemann at Genome Res. 11 (3), 422-435 (2001); Mus musculus, eukaryotic translation elongation factor 2 as described by Strausberg at http://www.ncbi.nlm.nih.gov/ entrez/viewer.cgi?val=13938071; M. musculus P311 as described by Studler et al. in Eur. J. Neurosci. 5 (6), 614-623 (1993); Homo sapiens E2a-Pb×1 associated protein (EB—1) as described by Fu et al. in Oncogene 18 (35), 4920-4929 (1999); Homo sapiens NADH dehydrogenase (ubiquinone) Fe-S protein 8 (23kD) (NADH—coenzyme Q reductase) as described by the NCBI annotation project at http://www.ncbi.nlm.nih.gov/entrez /query.fcgi?cmd=Retrieve &db=Nucleotide &list₁₃ uids=11525680&dopt=GenBank; Human Smad anchor for receptor activation (SARA) as described by Tsukazaki et al. in Cell 95 (6), 779-791 (1998); Homo sapiens AMSH as described by Tanaka et al. in J. Biol. Chem. 274 (27), 19129-19135 (1999); and Homo sapiens ATPase, H+ transporting, lysosomal (vacuolar proton pump), beta polypeptide, 56/58kD, isoform 2 (ATP6B2), as described by Bernasconi et al. in The Journal of biological chemistry. 265 (29), 17428-17431 (1990).

Co-administration of BSMR Effector(s) with Morphogenetic Protein(s)

Another embodiment of the invention is the combined administration of BSMR effector(s) with another protein or molecule that affects bone mineralization, either by synergy with BSMR or by an independent mechanism. In a preferred embodiment the other protein or molecule is selected from the group consisting of bone morphogenetic protein 2, another bone morphogenetic protein, hedgehog protein, endothelial growth factor, and TGF—beta. Preferably, multiple proteins or molecules are introduced transgenically via a single vector or multiple vectors, or are added together as pharmaceuticals. In many cases the pharmacological time of action of the other protein or molecule will differ, and the protein may be given later or, in many embodiments earlier than the BSMR effector.

In a particularly useful embodiment BMP2 is co-administered with one or more BSMR effectors. The inventors discovered that BMP2's effects on bone mineralization partly are mediated by the BSMR system and that adding a positive effector of that system enhances stimulation of bone mineralization. BMP2 may be added, for example, by injection to achieve a local concentration of between 1 ng and 100 ug per ml, more preferably between 10 ng and 10 ug per ml and yet more preferably between 50 ng and 1 ug per ml. In an advantageous embodiment BMP2 is added before addition of the effector. More preferably the BMP2 is added at least 24 hours prior to addition of a BSMR effector compound. In another embodiment a different bone morphogenetic protein such as BMP3 is used. Without wishing to be bound by any one theory of the invention, it is believed that BMP2 acts to increase synthesis of BSMR, allowing greater stimulation by an effector of BSMR that can react with more BSMR molecules.

In another embodiment of the invention a high level of a BSMR effector may be administered along with an LRP6 antagonist to prevent unnecessary stimulation of the LRP6 regulatory system. This is helpful for some BSMR effectors such as certain Wnt proteins that are known to bind LRP6. When using such a BSMR effector at high concentrations, a possible stimulation or overstimulation of LRP6 through cross-over binding to LRP6 is ameliorated by addition of the LRP6 antagonist. A preferred antagonist is a protein having one or more LRP6 binding sites. More preferably a specific LRP6 antagonist is added at the same time as the BSMR effector and in a suitable concentration to prevent excessive stimulation of LRP6 by the BSMR effector, for those BSMR effectors that interact with the LRP6 system. Preferably the LRP6 antagonist has an affinity for the LRP6 molecule that exceeds its affinity for BSMR (LRP5). More preferably the affinity for LRP6 is 2 times, more preferably 10 times and even more preferably at least 50 times higher than that for BSMR.

Targeted Delivery of BSMR Effector

In one embodiment of the invention a natural or synthetic BSMR effector is generated at or delivered to regions in the body that contain osteoblasts which express BSMR. A variety of delivery means for bone tissue, and particularly active growing bone tissue are known, including for example, injection into marrow, slow release from an agent placed in or near bone and the like are contemplated. In yet another embodiment, osteoblast cells and/or precursors of osteoblast cells are administered together with one or more BSMR effectors to a site where needed.

This embodiment of the invention arises from the discovery that BSMR and effectors of BSMR affect primarily regions of the body that contain osteoblasts. The data obtained and in Example 5 indicate the importance of this body distribution. In situ hybridization experiments in developing mice demonstrated that the clavicle, which is among the earliest skeletal elements to ossify during development, contains cells that actively express BSMR. The lateral half of the mouse clavicle ossifies, beginning on embryonic day 13.5 by membranous ossification, while the medial half ossifies later by endochondral ossification. It was found that skeletal expression of BSMR first occurs in cells that form the lateral part of the clavicle. These cells express other markers of skeletal differentiation, such as alkaline phosphatase and Cbfal as well, although the cells have not started to make detectable minerized matrix. In contrast, while the scapula anlage had already formed, it does not ossify until later and in fact was found to have no detectable BSMR expression at embryonic day 13.5. During embryonic day 16.5 when the diaphyseal bone collar is forming around the humerus, BSMR expression was noted in osteoblasts along the endosteal surface and not in growth plate chondrocytes (FIG. 3b). Analogous early and later stage bone forming regions in the human are readily appreciated by medical practitioners who may deliver an active BSMR effector to those region(s) according to this embodiment of the invention.

In addition to detecting osteoblastic expression in appendicular bones, expression was also noted in osteoblasts that line the developing calvaria. Postnatally, BSMR expression also was observed in trabecular and cortical osteoblasts in the appendicular skeleton in a 4 week-old mouse. LRP5 expression in the vitreal vasculature was assessed in embryonic day 17.5 mice as well. In contrast to humans, in which the primary vitreal vasculature has fully regressed by birth, the vasculature in mice persists at birth and regresses by the first postnatal month. However, no BSMR expression in mouse vitreous could be detected. These results thus indicate that higher amounts of BSMR may be found in areas of bone growth. In preferred embodiments a BSMR effector is targeted to a desired location in a body of a patient needing treatment by chemical or mechanical means. An effector, such as a molecule described herein may be targeted to bone tissue by the use of controlled-release compositions such as those described in WIPO publication WO 93/20859, which is incorporated herein by reference in its entirety.

One type of controlled release system utilizes mechanical attachment of polymer to bone. Briefly, a biodegradable polyester film, such as made from polylactic acid, polyglycolic acid, polydioxanone or polylactic acid/polyglycolic acid copolymer film, containing the effector is prepared and fabricated into a pin, plate, screw or the like for attachment to or insertion into bone. The compositions provide sustained release of the effector at the target site. Fifty percent PLA and fifty percent PGA films are preferred. These films further may include a carrier such as collagen, a recombinant collagen, Ossigel™ from Orquest Inc., Healos mineralized collagen matrix from Orquest Inc., albumin and/or a detergent such as polyoxyethylenesorbitan. When a protein such as albumin is included, the ratio of effector to the protein preferably in general is maintained between 0.01 and 10 milligrams effector per gram of protein. In principle, any substance that enhances polymer degradation, creates pores in the film or reduces adsorption of the effector(s) to the film can be used as a carrier. Human albumin is a particularly preferred carrier.

Materials that contain BSMR effectors are useful particularly as coatings for prosthetic devices and surgical implants. The films may, for example, be wrapped around the outer surfaces of surgical screws, rods, pins, plates and the like. Implantable devices of this type are routinely used in orthopedic surgery. The films can also be used to coat bone filling materials, such as hydroxyapatite blocks, demineralized bone matrix plugs, collagen matrices and the like. In general, a film or device as described herein is applied to the bone at the fracture site. Application is generally by implantation into the bone or attachment to the surface using standard surgical procedures.

In addition to the copolymers, effector substances and carriers noted above, the biodegradable films may include other active or inert components. Of particular interest are agents that promote tissue growth or infiltration. Representative agents include bone morphogenic protein(s) as for example described in U.S. Pat. No. 4,761,471 and PCT Publication WO 90/11366, osteogenin as for example described in Sampath et al., Proc. Natl. Acad. Sci. USA 84: 7109-7113, 1987, and NaF as described in Tencer et al., J. Biomed. Mat. Res. 23: 571-589, 1989.

To load the materials with effector, the BSMR effector(s) and a carrier may be applied to the (typically) film as powders or liquid solutions or incorporated into the film during film synthesis. For example, lyophilized effector and albumin may be uniformly dispersed over one surface of the film, and the film folded over. In the alternative, the substances may be applied as aqueous solutions (e.g., in phosphate buffered saline or distilled water), which are allowed to dry. Biodegradable materials containing the BSMR effector also may be molded into a variety of shapes as an implant according to procedures known in the art. Pins, plates, blocks, screws and the like can be fabricated for insertion into or attachment to bone at the site of a fracture or other defect.

Alternative methods for local delivery of BSMR effector and/or an additional agent useful for bone growth, regulation or repair include use of ALZET osmotic minipumps (Alza Corp. Palo Alto, Calif.); sustained release matrix materials such as those disclosed in Wang et al. (WO 90/11366); electrically charged dextran beads as disclosed in Bao et al. (WO 92/03125); collagen-based delivery systems, for example, as disclosed in Ksander et al. (Ann. Surg. 211(3):288-294, 1990); methylcellulose gel systems as disclosed in Beck et al. (J. Bone Min. Res. 6(11):1257-1265, 1991) and alginate-based systems as disclosed in Edelman et al. (Biomaterials, 12:619-626, 1991). Other methods well known in the art for sustained local delivery in bone include porous coated metal protheses that can be impregnated and solid plastic rods with therapeutic compositions incorporated within them.

Delivery of systemically administered compositions of the present invention may be enhanced by conjugating the effector to a targeting molecule. “Targeting molecule” in this context means a molecule that binds to (has affinity with) the tissue of interest. Examples of bone-targeting molecules include tetracyclines; calcein; bisphosphonates; polyaspartic acid; polyglutamic acid; aminophosphosugars; peptides known to be associated with the mineral phase of bone such as osteonectin, bone sialoprotein and osteopontin; bone specific antibodies; proteins with bone mineral binding domains and the like. See, for example, the disclosures of Bentz et al. (EP 0512844) and Murakami et al. (EP 0341961), the contents of which specifically are incorporated by reference in their entireties.

Screening Methods for Identifying Lead Pharmaceutical Agents as BSMR Effectors

In another embodiment of the invention, test substances such as peptides and synthetic chemicals are screened for their ability to interact with one or more components of the BSMR system. The screen may test for binding to BSMR directly, such as to an extracellular site on the protein, to an intracellular site on the protein, or to another protein that interacts with BSMR. Typically in a screen a test substance is incubated with a specific target of the BSMR system and binding is detected. The term “binding” in this context means that the association (determined by measuring an assocation constant) between test substance and the BSMR component is greater than the association between the same test substance and other protein generally. Preferably the binding between test substance and BSMR component is at least 10 times as strong and more preferably is at least 100 times as strong. Human serum albumin is preferred as a reference “other protein” because a large proportion of extracellular fluid protein is serum albumin.

In one embodiment a useful compound for screening preferably is a natural product, peptide, or other small compound capable of mimicking the effects of a naturally occuring regulator such as a Wnt protein. One preferred screening method uses the b—catenin/TCF signalling, another uses G—protein based biochemical reactions, and another uses downstream signalling pathway molecules based on the above identified reactive binding partners. The screening methods also may utilize BSMR target genes specific to osteoblasts or use the BSMR natural promoter to identify BSMR inducers.

A particularly desirable screening test is carried out by incubating one or more ligand binding sites (typically provided as a peptide, fusion protein, BSMR protein fragment or intact BSMR) in an aqueous solution with one or more test substances, and then determining binding between the test substance(s) and the ligand binding site. Binding assays are well known to the skilled artisan. For example, U.S. Pat. Nos. 5,976,814, 5,990,128 and 5,786,155 describe methods and tools for determining ligand binding, drug screening and associated techniques that are in use, and specifically are incorporated by reference in their entireties. In one embodiment, as explained in these patent documents, binding between a receptor expressed in intact cells and a test substance is used for screening.

In a preferred embodiment of testing and drug screening, a putative drug (chemical or protein) is desired that can bind to the extracellular portion of BSMR protein. Other types of compounds and proteins also may be detected by screening. Still others may be screened by assays that detect other biochemical behavior such as mitogenicity against cells that express the BSMR protein, induction of RNA or DNA synthesis, or synthesis of the BSMR protein. In another screening methodology embodiment an intact cell that expresses the BSMR protein is used and a biochemical or morphological event such as an enzyme reaction coupled to light production is detected to indicate that a test compound is a BSMR effector and thus a candidate as a lead compound for drug development.

A ligand that demonstrates specific binding in such a binding assay described here will, according to many embodiments, modulate the receptor system. Specific BSMR effectors such as extracellular ligands may be discovered this way and used as pharmaceuticals to increase bone strength and mineralization. Other ligands of course can be discovered and are useful to reduce or minimize bone growth by affecting the BSMR regulatory system in different direction.

In an embodiment, a compound for screening may be a natural product, peptide, or other small compound capable of mimicking the effects of a naturally occuring regulator such as a Wnt protein. One preferred screening method uses the b—catenin/TCF signalling, another uses G—protein based biochemical reactions, and another uses downstream signalling pathway molecules based on the above identified reactive binding partners. The screening methods also may utilize BSMR target genes specific to osteoblasts or use the BSMR natural promoter to identify BSMR inducers.

In another embodiment useful for discovery of a bone mineralization BSMR effector, a Wnt gene, Wnt polypeptide or derivative thereof may be prepared as described for example in U.S. Pat. No. 6,159,462. This patent describes the preparation of Wnt Genes and Gene Products, particularly by recombinant production of Wnt genes and gene products through culturing cells transformed with a vector containing Wnt polypeptide-encoding nucleic acid, and recovering the polypeptide from the cell culture.

In the procedure a DNA encoding a Wnt polypeptide is obtained from any cDNA library prepared from tissue believed to possess the Wnt polypeptide mRNA and that can express the DNA at a detectable level. For example, Wnt polypeptide DNA can be conveniently obtained from a cDNA library prepared from mammalian fetal liver or fetal brain. The Wnt polypeptide-encoding gene may also be obtained from a genomic library or by oligonucleotide synthesis. Libraries are screened with probes (such as antibodies to the Wnt polypeptide, or oligonucleotides of about 20-80 bases) designed to identify the gene of interest or the protein encoded by it. Screening the cDNA or genomic library with the selected probe may be conducted using standard procedures as described in chapters 10-12 of Sambrook et al., Molecular Cloning: A Laboratory Manual (New York: Cold Spring Harbor Laboratory Press, 1989). An alternative means to isolate the gene encoding Wnt polypeptide is to use PCR methodology as described in section 14 of Sambrook et al.

Amino acid sequence variants of Wnt polypeptide useful as BSMR effectors according to this embodiment can be prepared by introducing appropriate nucleotide changes into the Wnt polypeptide DNA, or by synthesis of the desired Wnt polypeptide. Such variants represent insertions, substitutions, and/or specified deletions of, residues within or at one or both of the ends of the amino acid sequence of a naturally occurring human Wnt polypeptide. Preferably, these variants represent insertions and/or substitutions within or at one or both ends of the mature sequence, and/or insertions, substitutions and/or specified deletions within or at one or both of the ends of the signal sequence of the Wnt polypeptide. Any combination of insertion, substitution, and/or specified deletion is made to arrive at the final construct, provided that the final construct possesses the desired biological activity as defined herein. The amino acid changes may include post-translational processing of the Wnt polypeptide. Such processing may involve changing the number or position of glycosylation sites, altering the membrane anchoring characteristics, and/or altering the intracellular location of the Wnt polypeptide by inserting, deleting, or otherwise affecting the leader sequence of the Wnt polypeptide.

Variations in the native sequence as described above can be made using any of the techniques and guidelines for conservative and non-conservative mutations as for example set forth in U.S. Pat. No. 5,364,934. These include oligonucleotide-mediated (site-directed) mutagenesis, alanine scanning, and PCR mutagenesis. The nucleic acid (e.g., cDNA or genomic DNA) encoding the Wnt polypeptide is inserted into a replicable vector for further cloning (amplification of the DNA) or for expression. Many vectors are available for this use. The vector components generally include, but are not limited to, one or more of the following: a signal sequence, an origin of replication, one or more marker genes, an enhancer element, a promoter, and a transcription termination sequence.

This same general scheme may be used for discovery and modification of other polypeptides that resemble natural components of the BSMR system as discovered and described herein. In many embodiments, however, pharmaceutical lead compounds are screened for their abilities to bind directly to the extracellular region of BSMR. Specific binding to this receptor is particularly desirable for pharmacological intervention to promote improved bone status because of decreased side effects from interacting with a specific (i.e. BSMR) receptor. In one desirable embodiment a compound is selected for its effect on bone metabolism by looking for preferential binding to BSMR over LRP6. This differential binding is very useful for drug screening because LRP6 is similar in structure to BSMR and is expected to at bind at least some of the BSMR effectors. By choosing a lead compound that binds preferably to BSMR over LRP6, non-specific side effects of the pharmaceutical may be minimized. The phrase “that binds preferentially to” in this context means that the binding is at least 2 times, preferably at least 5 times, more preferably at least 20 times, yet more preferably at least 100 times and even more preferably more than 500 times as strong as determined by measuring and calculating an association constant at pH 7.0 at 37 degree C. in aqueous solution or suspension.

Genetic Therapy Based on Expression of Extracellular and/or Intracellular Ligands

In a preferred embodiment long term control of BMSR protein is achieved by transgenic expression of one or more of the ligands listed above. A wide range of methods are useful for transgenic expression, including, for example, introduction of nucleic acid by a virus and introduction by vesicles. Preferably the transgenic expression occurs primarily in the bone. This is best carried out by administration to the bone or by selectively activating gene in bone material compared with non-bone material.

Most preferably bone specific gene expression is carried out by making a vector for a gene of the extracellular ligand that directs bone specific gene expression. For example, U.S. Pat. No. 5,948,951 issued Sep. 7, 1999 to Gardiner et al. describes an upstream regulatory element obtained from the osteocalcin gene and the VDRE gene and which can be combined with a transgene to obtain bone specific expression. The contents of this patent in particular are expressly incorporated by reference in their entirety. Most preferred in this context is a vector useful for improving bone strength and mineralization that comprises both the upstream positive regulatory element of osteocalcin and at least one gene that encodes an effector of BSMR such as an extracellular ligand of BSMR.

In another desirable embodiment an extracellular antagonist alters bone strength and mineralization by interfering with binding of a stimulatory ligand such as one of the stimulatory ligands shown in FIG. 13, to the BSMR protein. For example, dkk—1, dkk—2, dkk—3, RAP, sFRP and human analogues of these proteins can interfere and have various degrees of activity. In a preferred embodiment a polynucleic acid encoding a gene for a naturally occuring antagonist of BSMR activity is added in an antisense format such that an antisense strand forms and binds to messenger RNA, thus blocking translation and/or triggering nuclease destruction of the RNA. Recombination using antisense technology has previously been used to inhibit expression of specific gene products in mammalian cell lines (Kasid et al., Science 243:1354-1356 1989; Khoka et al., Science 243:947-950 1989; Izant et al., Science 229:345-352 1985) including some retroviruses; (von Ruden et al., J. Virol. 63:677-682 1989; and Chang et al., J. Virol. 61:921-924 1987). Each of these methods, and later developed methods specifically are contemplated for this embodiment of the invention.

Dosage, Composition and Formulation of Pharmaceuticals for Prophylaxis and Therapy for Increased Bone Strength and Density

Pharmaceutical compositions contemplated for embodiments of the invention comprise ingredients in an “effective amount” to achieve increased bone strength. Determination of the effective amounts is well within the capability of those skilled in the art, especially in light of the detailed disclosure provided herein. A therapeutically effective dose refers to that amount of the compound that results in amelioration of symptoms or a measurable increase in bone strength.

Generally speaking, an “effective amount” of a composition is that amount which produces a statistically significant effect. When determined by effects on stimulating the growth of osteoblast cells in vitro, it is generally desirable to produce an increase in growth of at least 50%, as measured by incorporation of tritiated thymidine, as compared to cells grown in the absence of added effector. For therapeutic uses, an “effective amount” is the amount of the composition comprising the effector required to provide a clinically significant increase in healing rates in fracture repair, reversal of bone loss in osteoporosis, stimulation and/or augmentation of bone formation in fracture non-unions and distraction osteogenesis, increase and/or acceleration of bone growth into prosthetic devices and repair of dental defects. Such amounts will depend, in part, on the particular condition to be treated and other factors evident to those skilled in the art. For example in osteoporosis, increase in bone formation is manifested as a statistically significant difference in bone mass between treatment and control groups. This can be seen as, for example, a 10-20% or more increase in bone mass. Other measurements of clinically significant increases in healing may include, for example, tests for breaking strength and tension, breaking strength and torsion, 4-point bending and other biomechanical tests well known to those skilled in the art. General guidance for treatment regimens is obtained from experiments carried out in animal models of the disease of interest.

Toxicity and therapeutic efficacy of such compounds also may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD50 and ED50. Compounds which exhibit high therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p1). Dosage amount and interval may be adjusted individually to provide plasma levels of the active moiety which are sufficient to maintain the desired effects.

In cases of local administration or selective uptake, the effective local concentration of the drug might not be related to plasma concentration. The amount of composition administered will, of course, be dependent on the subject being treated, on the subject's weight, the severity of the affliction, the manner of administration and the judgment of the prescribing physician. The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes. Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agent(s) of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art. For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner. For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A preferred pharmaceutical carrier for the hydrophobic compounds of the invention is a co-solvent system comprising benzyl alcohol, a nonpolar surfactant, a water-miscible organic polymer, and an aqueous phase. Naturally, the proportions of a co-solvent system may be varied considerably without destroying its solubility and toxicity characteristics. Furthermore, the identity of the co-solvent components may be varied. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections. Alternately, one may administer the compound in a local rather than systemic manner, for example, via injection of the compound directly into an affected area, often in a depot or sustained release formulation. Furthermore, one may administer the drug in a targeted drug delivery system, for example, in a liposome coated with an antibody specific for affected cells. The liposomes will be targeted to and taken up selectively by the cells.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

DNA-Based Diagnosis of Bone Strength and Mineralization

One embodiment of the invention is the application of BSMR DNA sequence information presented herein for genetic testing, carrier detection and prenatal diagnosis for genetic predisposition to weak bone strength and to OPS. Individuals carrying mutations in the BSMR gene (OPS disease carrier or patients or any individual with a BSMR mutation leading to poor bone strength) may be detected at the DNA level using any of a variety of techniques. For such a diagnostic procedure, a biological sample of the subject, which biological sample contains either DNA or RNA derived from the subject, is assayed for the presence of a mutant BSMR gene. Suitable biological samples include samples containing genomic DNA or RNA obtained from body cells, such as those present in peripheral blood, urine, saliva, tissue biopsy, surgical specimen, amniocentesis samples and autopsy material. The detection in the biological sample of either a mutant BSMR gene or a mutant BSMR RNA may be performed by a number of methodologies, as outlined below.

A preferred embodiment of such detection techniques is the polymerase chain reaction amplification of reverse transcribed RNA (RT-PCR) of RNA isolated from bone marrow cells or other cells followed by direct DNA sequence determination of the products. The presence of one or more nucleotide difference between the obtained sequence and the DNA sequences presented in FIG. 2, and especially, differences in the ORF portion of the nucleotide sequence are taken as indicative of a potential BSMR gene mutation.

The detection of specific DNA mutations may be achieved by methods such as hybridization using specific oligonucleotides (Wallace et al. (1986). Cold Spring Harbor Symp. Quant. Biol. 51:257-261), direct DNA sequencing (Church and Gilbert (1988). Proc. Natl. Acad. Sci. USA 81:1991-1995), the use of restriction enzymes (Flavell et al. (1978). Cell 15:25), discrimination on the basis of electrophoretic mobility in gels with denaturing reagent (Myers and Maniatis (1986). Cold Spring Harbor Symp. Quant. Biol. 51:275-284), RNase protection (Myers et al. (1985). Science 230:1242), chemical cleavage (Cotton et al. (1985). Proc. Natl. Acad. Sci. USA 85:4397-4401), and the ligase-mediated detection procedure (Landegren et al., 1988).

Oligonucleotides specific to normal or mutant sequences are chemically synthesized using commercially available machines, labelled radioactively with isotopes (such as ³²P) or non-radioactively (with tags such as biotin (Ward and Langer et al. (1981). Proc. Natl. Acad. Sci. USA 78:6633-6657), and hybridized to individual DNA samples immobilized on membranes or other solid supports by dot-blot or transfer from gels after electrophoresis. The presence or absence of these specific sequences are determined optically by methods such as autoradiography or fluorometric (Landegren, et al., 1989) or calorimetric reactions (Gebeyehu et al. (1987). Nucleic Acids Res. 15:4513-4534).

Sequence differences between normal and mutant forms of the gene may also be revealed by the direct DNA sequencing method of Church and Gilbert (1988). Cloned DNA segments may be used as probes to detect specific DNA segments. The sensitivity of this method is greatly enhanced when combined with PCR (Wrichnik et al., 1987; Wong et al., 1987; Stoflet et al. (1988). Science 239:491-494). In this approach, a sequencing primer which lies within the amplified sequence is used with double-stranded PCR product or single-stranded template generated by a modified PCR. The sequence determination is performed by conventional procedures with radiolabeled nucleotides or by automatic sequencing procedures with fluorescent tags.

Sequence alterations occasionally may generate fortuitous restriction enzyme recognition sites or may eliminate existing restriction sites. Changes in restriction sites are revealed by the use of appropriate enzyme digestion followed by conventional gel-blot hybridization (Southern (1975). J. Mol. Biol. 98:503). DNA fragments carrying the site (either normal or mutant) are detected by their reduction in size or increase of corresponding restriction fragment numbers. Genomic DNA samples may also be amplified by PCR prior to treatment with the appropriate restriction enzyme; fragments of different sizes are then visualized under UV light in the presence of ethidium bromide after gel electrophoresis.

Genetic testing based on DNA sequence differences may be achieved by detection of alteration in electrophoretic mobility of DNA fragments in gels with or without denaturing reagent. Small sequence deletions and insertions can be visualized by high-resolution gel electrophoresis. For example, a PCR product with small deletions is clearly distinguishable from a normal sequence on an 8% non-denaturing polyacrylamide gel (WO 91/10734,) Nagamine et al. Am. J. Hum. Genet. 45:337-339 (1989). DNA fragments of different sequence compositions may be distinguished on denaturing formamide gradient gels in which the mobilities of different DNA fragments are retarded in the gel at different positions according to their specific “partial-melting” temperatures (Myers et al. (1985). Science 230:1242). Alternatively, a method of detecting a mutation comprising a single base substitution or other small change could be based on differential primer length in a PCR. For example, an invariant primer could be used in addition to a primer specific for a mutation. The PCR products of the normal and mutant genes can then be differentially detected in acrylamide gels.

In addition to conventional gel-electrophoresis and blot-hybridization methods, DNA fragments may also be visualized by methods where the individual DNA samples are not immobilized on membranes. The probe and target sequences may be both in solution, or the probe sequence may be immobilized (Saiki et al. Proc. Nat. Acad. Sci. USA 86:6230-6234 (1989). A variety of detection methods, such as autoradiography involving radioisotopes, direct detection of radioactive decay (in the presence or absence of scintillant), spectrophotometry involving calorigenic reactions and fluorometry involved fluorogenic reactions, may be used to identify specific individual genotypes.

Other more recent methods to detect genetic polymorphism are useful for embodiments of the invention. A particularly good example is that disclosed in U.S. Pat. No. 5,856,104 issued to Chen et al. Jan. 5, 1999, the contents of which specifically are incorporated by reference in its entirety.

If more than one mutation is frequently encountered in the BSMR gene, a system capable of detecting such multiple mutations would be desirable. For example, a PCR with multiple, specific oligonucleotide primers and hybridization probes may be used to identify all possible mutations at the same time (Chamberlain et al. (1988). Nucl. Acids Res. 16:1141-1155 (1988)). The procedure may involve immobilized sequence-specific oligonucleotides probes (Saiki et al. Proc. Nat. Acad. Sci. USA 86:6230-6234) (1989).

DNA Vectors Useful for Bone Therapy and Prophylaxis

Other embodiments of the invention provide recombinant DNA vectors comprising the disclosed DNA molecules useful for modulating bone strength and mineralization, and transgenic host cells containing such recombinant vectors.

With the provision of the BSMR cDNA and the understanding of the role and mechanisms of the coded protein in bone strength and mineralization, vectors that contain BSMR DNA may be used for diagnosis and therapy. Furthermore, the DNA sequence of the BSMR cDNA and polymorphic or mutated BSMR cDNAs isolated from OPS patients and other patients can be manipulated in studies to further understand the expression of the gene and the function of its product. In this way, further ligands usful for embodiments of the invention and mechanisms involved in control of mineralization can be discovered.

The mutant versions of BSMR DNA isolated to date and other nucleic acid sequences which may be isolated based upon information contained herein, may be used to detect alteration in expression patterns in terms of relative quantities, tissue specificity and functional properties of the encoded mutant BSMR protein in individuals to determine their bone mineralizing status. Partial or full-length DNA sequences, which encode BSMR may be ligated into bacterial expression vectors. Methods for expressing large amounts of protein from a cloned gene that is introduced into Escherichia coli (E. coli) may be utilized to purify, localize and study the function of proteins involved in the regulation. For example, fusion proteins consisting of amino terminal peptides encoded by a portion of the E. coli lacZ or trpE gene linked to BSMR proteins may be used to prepare polyclonal and monoclonal antibodies against these proteins. Thereafter, these antibodies may be used to purify proteins by immunoaffinity chromatography, in diagnostic assays to quantitate the levels of protein and to localize proteins in tissues and individual cells by immunofluorescence.

Intact native protein also may be produced in E. coli in large amounts for functional studies. Native proteins can be produced in bacteria by placing a strong, regulated promoter and an efficient ribosome binding site upstream of the cloned gene. If low levels of protein are expressed, additional steps may be taken to increase protein production; if high levels of protein are expressed, purification is easier. Suitable methods are presented in Sambrook et al. (1989). In Molecular Cloning: A Laboratory Manual, Cold Spring Harbor, N.Y. and are well known in the art. Often, proteins expressed at high levels are found in insoluble inclusion bodies. Methods for extracting proteins from these aggregates are described by Sambrook et al. (1989). In Molecular Cloning: A Laboratory Manual, Cold Spring Harbor, N.Y. Vector systems suitable for the expression of lacZ fusion genes include the pUR series of vectors (Ruther and Muller-Hill (1983). EMBO J. 2:1791), pEX1-3 (Stanley and Luzio (1984). EMBO J. 3:1429) and pMR100 (Gray et al. (1982). Proc. Natl. Acad. Sci. USA 79:6598). Vectors suitable for the production of intact native proteins include pKC30 (Shimatake and Rosenberg (1981). Nature (London) 292:128), pKK177-3 (Amann and Brosius (1985). Gene 40:183.) and pET-3 (Studiar and Moffatt (1986). J. Mol. Biol. 189:113).

Methods and plasmid vectors for producing fusion proteins and intact native proteins in bacteria are described in Sambrook et al. (1989). In Molecular Cloning: A Laboratory Manual, Cold Spring Harbor, N.Y. (ch. 17, herein incorporated by reference). Such fusion proteins may be made in large amounts, are easy to purify, and can be used to elicit antibody response. BSMR fusion proteins may be isolated from protein gels, lyophilized, ground into a powder and used as an antigen. The DNA sequence can also be transferred from its existing context in pREP4 to other cloning vehicles, such as other plasmids, bacteriophages, cosmids, animal viruses and yeast artificial chromosomes (YACs) (Burke et al. (1987). Science 236:806-812). These vectors may then be introduced into a variety of hosts including somatic cells, and simple or complex organisms, such as bacteria, fungi (Timberlake and Marshall (1989). Science 244:1313-1317), invertebrates, plants (Gasser and Fraley (1989). Science 244:1293), and pigs (Pursel et al. (1989). Science 244:1281-1288), which cell or organisms are rendered transgenic by the introduction of the heterologous BSMR cDNA.

In one embodiment of the invention, affinity of the BSMR protein to a bone mineralizing ligand is increased by adding a peptide segment that has one or more binding sites to the protein. Preferably the added segment is a copy of one of the three LDLR repeat ligand binding regions from a native protein. In another embodiment, a portion of the amino terminal end of this protein is deleted. In one embodiment between 25% and 50% of the extracellular portion from the amino terminal end is deleted and in another embodiment at least 50% is deleted. After deletion, ligands can more easily bind to the ligand binding regions that are adjacent to the membrane surface.

For expression in mammalian cells, the cDNA sequence may be ligated to heterologous promoters, such as the simian virus (SV)40, promoter in the pSV2 vector (Mulligan and Berg, 1981), and introduced into cells, such as monkey COS-1 cells (Gluzman (1981). Cell 23:175-182), to achieve transient or long-term expression. The stable integration of the chimeric gene construct may be maintained in mammalian cells by biochemical selection, such as neomycin (Southern and Berg (1982). J. Mol. Appl. Genet. 1:327-341) and mycophoenolic acid (Mulligan and Berg, 1981).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, sitedirected sequence-alteration via single-stranded bacteriophage intermediate or with the use of specific oligonucleotides in combination with PCR.

The cDNA sequence (or portions derived from it) or a mini gene (a cDNA with an intron and its own promoter) is introduced into eukaryotic expression vectors by conventional techniques. These vectors are designed to permit the transcription of the cDNA eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (Mulligan et al., 1981; Gorman et al. (1982). Proc. Natl. Acad. Sci. USA 78:6777-6781). The level of expression of the cDNA can be manipulated with these types of vector, either by using promoters that have different activities (for example, the baculovirus pAC373 can express cDNAs at high levels in S. frugiperda cells (Summers and Smith (1985). In Genetically Altered Viruses and the Environment, Fields et al. (Eds.) 22:319-328, Cold Spring Harbor Laboratory Press, Cold Spring Harbor, N.Y.) or by using vectors that contain promoters amenable to modulation, for example, the glucocorticoid-responsive promoter from the mouse mammary tumor virus (Lee et al. (1982). Nature 294:228). The expression of the cDNA can be monitored in the recipient cells 24 to 72 hours after introduction (transient expression).

In addition, some vectors contain selectable markers such as the gpt (Mulligan and Berg, 1981) or neo (Southern and Berg (1982). J. Mol. Appl. Genet. 1:327-341) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (Schafner (1980). Proc. Natl. Acad. Sci. USA 77:2163-2167). Proc. Natl. Acad. Sci. USA 77:2163-2167). Mol. Cell Biol. 1:486) or Epstein-Barr (Sugden et al. (1985). Mol. Cell Biol. 5:410). Such episomal vectors are exemplified by the pREP4 Epstein-Barr virus vector in which the cDNA library described in Example 2 herein was constructed. Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al. (1978). J. Biol. Chem. 253:1357).

The transfer of DNA into eukaryotic, in particular human or other mammalian cells, is now a conventional technique. The vectors are introduced into the recipient cells as pure DNA (transfection) by, for example, precipitation with calcium phosphate (Graham and vander Eb (1973). Virology 52:466) or strontium phosphate (Brash et al. (1987). Mol. Cell Biol. 7:2013), electroporation (Neumann et al. (1982). EMBO J 1:841), lipofection (Felgner et al. (1987). Proc. Natl. Acad. Sci. USA 84:7413), DEAE dextran (McCuthan et al. (1968). J. Natl. Cancer Inst. 41:351), microinjection (Mueller et al. (1978). Cell 15:579), protoplast fusion (Schafner (1980). Proc. Natl. Acad. Sci. USA 77:2163-2167), or pellet guns (Klein et al., 1987). Alternatively, the cDNA can be introduced by infection with virus vectors. Systems are developed that use, for example, retroviruses (Bernstein et al. (1985). Gen. Engr'g 7:235), adenoviruses (Ahmad et al. (1986). J. Virol. 57:267.), or Herpes virus (Spaete et al. (1982). Cell 30:295).

These eukaryotic expression systems can be used for studies of the BSMR gene and mutant forms of this gene, the BSMR protein and mutant forms of this protein. Such uses include, for example, the identification of regulatory elements located in the 5' region of the BSMR gene on genomic clones that can be isolated from human genomic DNA libraries using the information contained in the present invention. The eukaryotic expression systems may also be used to study the function of the normal complete protein, specific portions of the protein, or of naturally occurring or artificially produced mutant proteins. Naturally occurring mutant proteins exist in patients with BSMR, while artificially produced mutant proteins can be designed by site directed mutagenesis as described above. These latter studies may probe the function of any desired amino acid residue in the protein by mutating the nucleotide coding for that amino acid.

Using the above techniques, the expression vectors containing the BSMR gene sequence or fragments or variants or mutants thereof can be introduced into human cells, mammalian cells from other species or non-mammalian cells as desired. The choice of cell is determined by the purpose of the treatment. Generally, transfer into human bone marrow cells is preferred.

The following is provided as one exemplary method to express BSMR polypeptide from the cloned BSMR cDNA sequences in mammalian cells. Cloning vector pXTI, commercially available from Stratagene, contains the Long Terminal Repeats (LTRs) and a portion of the GAG gene from Moloney Murine Leukemia Virus. The position of the viral LTRs allows highly efficient, stable transfection of the region within the LTRs. The vector also contains the Herpes Simplex Thymidine Kinase promoter (TK), active in embryohal cells and in a wide variety of tissues in mice, and a selectable neomycin gene conferring G418 resistance. Two unique restriction sites Bg1II and XhoI are directly downstream from the TK promoter. BSMR cDNA, including the entire open reading frame for the BSMR protein and the 3′ untranslated region of the cDNA is cloned into one of the two unique restriction sites downstream from the promoter.

The ligated product is transfected into mouse NIH 3T3 cells using Lipofectin (Life Technologies, Inc.) under conditions outlined in the product specification. Positive transfectants are selected after growing the transfected cells in 600.mu.g/ml G418 (Sigma, St. Louis, Mo.). The protein is released into the supernatant and may be purified by standard immunoaffinity chromatography techniques using antibodies raised against the BSMR protein, as described below.

Expression of the BSMR protein in eukaryotic cells may also be used as a source of proteins to raise antibodies. The BSMR protein may be extracted following release of the protein into the supernatant as described above, or, the cDNA sequence may be incorporated into a eukaryotic expression vector and expressed as a chimeric protein with, for example, beta-globin. Antibody to .beta.—globin is thereafter used to purify the chimeric protein. Corresponding protease cleavage sites engineered between the .beta.—globin gene and the cDNA are then used to separate the two polypeptide fragments from one another after translation. One useful expression vector for generating .beta.—globin chimeric proteins is pSG5 (Stratagene). This vector encodes rabbit .beta.—globin.

The recombinant cloning vector, according to this invention, then comprises the selected DNA of the DNA sequences of this invention for expression in a suitable host. The DNA is operatively linked in the vector to an expression control sequence in the recombinant DNA molecule so that the BSMR polypeptide can be expressed. The expression control sequence may be selected from the group consisting of sequences that control the expression of genes of prokaryotic or eukaryotic cells and their viruses and combinations thereof. The expression control sequence may be specifically selected from the group consisting of the lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3—phosphoglycerate kinase, the promoters of yeast acid phosphatase, the promoter of the yeast alpha-mating factors and combinations thereof.

Retroviruses have been considered the preferred vector for experiments in gene therapy, with a high efficiency of infection and stable integration and expression (Orkin et al. (1988). Prog. Med. Genet. 7:130). The full length BSMR gene or DNA can be cloned into a retroviral vector and driven from either its endogenous promoter or from the retrovital LTR (long terminal repeat). Other viral transfection systems may also be utilized for this type of approach, including Adeno-Associated virus (AAV) (McLaughlin et al. (1988). J. Virol. 62:1963), Vaccinia virus (Moss et al. (1987). Annu. Rev. Immunol. 5:305), Bovine Papilloma virus (Rasmussen et al. (1987). Methods Enzymol. 139:642) or members of the herpesvirus group such as Epstein-Barr virus (Margolskee et al. (1988). Mol. Cell. Biol. 8:2837-2847).

The mouse is an extremely useful experimental organism for experiments with transgenic technology. The cloning of the mouse BSMR homologue permits generation of a mouse model for BSMR by targeted gene replacement in mouse embryonic stem cells (Sedivy and Joyner (1992). In Gene Targeting, W. H. Freeman and Company, New York). This in turn, will facilitate the study of the abnormal developmental processes leading to the pleiotropic phenotype of osteopathic disease.

The host cell, which may be transfected with the vector of this invention, may be selected from the group consisting of E. coli, Pseudomonas, Bacillus subtilis, Bacillus stearothermophilus or other bacilli; other bacteria; yeast; fungi; insect; mouse or other animal; or plant hosts; or human tissue cells.

It is appreciated that for mutant or variant DNA sequences, similar systems are employed to express and produce the mutant product.

Diagnosis of Bone Status Predisposition using the BSMR Gene Sequence

One embodiment of the invention is a screening method to determine if a subject carries a mutant BSMR gene and or to determine bone strength and mineralization activity. The method comprises the steps of: providing a biological sample obtained from the subject, which sample includes DNA or RNA, providing an assay for detecting in the biological sample the presence of at least one member from the group consisting of a mutant BSMR gene and a mutant BSMR RNA. A preferred embodiment of this method is described wherein the assay comprises a method selected from the group consisting of: hybridization with oligonucleotides; PCR amplification of the BSMR gene or a part thereof using oligonucleotide primers; RT-PCR amplification of the BSMR RNA or a part thereof using oligonucleotide primers, and direct sequencing of the BSMR gene of the subject's genome using oligonucleotide primers. When the availability of intron sequence data from the splice sites of the human BSMR gene and polymerase chain reactions for the amplification of these sequences from genomic DNA, as provided by this invention, will permit the analysis of these regions for potential splice site mutations. Furthermore, the efficiency of these molecular genetic methods should permit a more rapid classification of OPS patients than is possible with the labor intensive method of classical complementation analysis.

A further aspect of the present invention is a method for screening a subject to assay for the presence of a mutant BSMR gene comprising the steps of: providing a biological sample of the subject which sample contains cellular proteins and providing an immunoassay for determining the bone strength and mineralization stimulation quality of BSMR protein in the biological sample, based on differential binding to antibodies. Most preferred is an embodiment that used monoclonal antibodies that react with ligand binding site(s) on the extracellular portion of the BSMR protein. The generation of monoclonal antibodies using peptide, chimeric protein, or entire intact BSMR protein that contains at least one LDLR repeat ligand binding site sequence of the BSMR protein uses routine methodology.

In further embodiments, a subject is screened to determine his or her polymorphism for the BSMR gene. This information is used to predict propensity to a heavy or light bone mass. Various techniques for assaying genetic polymorphism are known, as exemplified in U.S. Pat. Nos. 6,074,831; 5,364,759; 5,614,364; and 5,856,104, the contents of which specifically are incorporated by reference in their entireties, particularly with respect to their descriptions on how to detect polymorphism of a gene.

Another embodiment of the invention is an immunoassay for detecting predisposition for heavy bone mass, comprising antibodies that specifically bind BSMR protein, wherein the antibodies are selected from the group consisting of monoclonal antibodies and polyclonal antibodies. Techniques for using the antibodies in a kit (that is, in a package with an instructional chart or package insert) are routinue and are contemplated as further embodiments of the invention.

Through the manipulation of the nucleotide sequence of the human or murine cDNAs provided by this invention by standard molecular biology techniques, variants of the OPS and OPS proteins may be made which differ in precise amino acid sequence from the disclosed proteins yet which maintain the essential characteristics of the OPS and OPS proteins or which are selected to differ in some characteristics from these proteins. Such variants are another aspect of the present invention.

Nucleic Acids Useful for Embodiments of the Invention

A large variety of isolated human BSMR cDNA sequences, animal, and particularly mouse homologues of this sequence and partial sequences that hybridize with the BMG gene are useful for detecting an individual's tendency for bone strength and mineralization and for altering bone strength and mineralization according to embodiments of the invention. Representative nucleic acid sequences in this context have been shown in the figures. Still further variants of these sequences and sequences of nucleic acids that hybridize with those shown in the figures also are comprehended for use in detection and alteration of mineralization status/tendency, as are the genomic genes from which these cDNAs are derived.

Many of the contemplated variant DNA molecules include those created by standard DNA mutagenesis techniques, for example, M13 primer mutagenesis. Details of these techniques are provided in Sambrook et al. (1989). In Molecular Cloning: A Laboratory Manual, Cold Spring Harbor, N.Y.. By the use of such techniques, variants may be created which differ in minor ways from those disclosed. DNA molecules and nucleotide sequences which are derivatives of those specifically disclosed herein and which differ from those disclosed by the deletion, addition or substitution of nucleotides while still encoding a protein which possesses the functional characteristic of the BSMR protein are comprehended by this invention. Also within the scope of this invention are small DNA molecules which are derived from the disclosed DNA molecules. Such small DNA molecules include oligonucleotides suitable for use as hybridization probes or polymerase chain reaction (PCR) primers. As such, these small DNA molecules will comprise at least a segment of an BSMR cDNA molecule or the BSMR gene and, for the purposes of PCR, will comprise at least a 10-15 nucleotide sequence and, more preferably, a 15-30 nucleotide sequence of the BSMR cDNA or the BSMR gene. DNA molecules and nucleotide sequences which are derived from the disclosed DNA molecules as described above may also be defined as DNA sequences which hybridize under stringent conditions to the DNA sequences disclosed, or fragments thereof.

Hybridization conditions corresponding to particular degrees of stringency vary depending upon the nature of the hybridization method of choice and the composition and length of the hybridizing DNA used. Generally, the temperature of hybridization and the ionic strength (especially the sodium ion concentration) of the hybridization buffer will determine the stringency of hybridization. Calculations regarding hybridization conditions required for attaining particular degrees of stringency are discussed by Sambrook et al. (1989). In Molecular Cloning: A Laboratory Manual, Cold Spring Harbor, N.Y., chapters 9 and 11, herein incorporated by reference. By way of illustration only, a hybridization experiment may be performed by hybridization of a DNA molecule (for example, a deviation of the BSMR cDNA) to a target DNA molecule (for example, the BSMR cDNA) which has been electrophoresed in an agarose gel and transferred to a nitrocellulose membrane by Southern blotting (Southern (1975). J. Mol. Biol. 98:503), a technique well known in the art and described in Sambrook et al. (1989). In Molecular Cloning: A Laboratory Manual, Cold Spring Harbor, N.Y. Hybridization with a target probe labeled with isotopic P (32) labelled-dCTP generally is carried out in a solution of high ionic strength such as 6 times SSC at a temperature that is 20-25 degrees Celsius, below the melting temperature, Tₘ, described below. For such Southern hybridization experiments where the target DNA molecule on the Southern blot contains 10 ng of DNA or more, hybridization is typically carried out for 6-8 hours using 1-2 ng/ml radiolabeled probe (of specific activity equal to 10⁹ CPM/mug or greater). Following hybridization, the nitrocellulose filter is washed to remove background hybridization. The washing conditions should be as stringent as possible to remove background hybridization but to retain a specific hybridization signal. The term “T_{m”} represents the temperature above which, under the prevailing ionic conditions, the radiolabeled probe molecule will not hybridize to its target DNA molecule. The Tₘ of such a hybrid molecule may be estimated from the following equation (Bolton and McCarthy (1962). Proc. Natl. Acad. Sci. USA 48:1390): Tₘ=81.5 degrees C.−16.6(log₁₀ of sodium ion concentration)+0.41(%G+C)−0.63(% formamide)−(600/1) where 1=the length of the hybrid in base pairs. This equation is valid for concentrations of sodium ion in the range of 0.01M to 0.4M, and it is less accurate for calculations of Tₘ in solutions of higher sodium ion concentration. The equation also is valid for DNA having G+C contents within 30% to 75%, and also applies to hybrids greater than 100 nucleotides in length. The behavior of oligonucleotide probes is described in detail in Ch. 11 of Sambrook et al. (1989). In Molecular Cloning: A Laboratory Manual, Cold Spring Harbor, N.Y.

Thus, by way of example of a 150 base pair DNA probe derived from the first 150 base pairs of the open reading frame of the BSMR cDNA (with a % GC=45%), a calculation of hybridization conditions required to give particular stringencies may be made as follows:

For this example, it is assumed that the filter will be washed in 0.3 X SSC solution following hybridization, thereby sodium ion =0.045M; % GC=45%; Formamide concentration=01=150 base pairs (EQU1) and so Tₘ=74.4 degrees C.

The Tₘ of double-stranded DNA decreases by 1-1.5 degrees C. with every 1% decrease in homology (Bonner et al. (1973). J. Mol. Biol. 81:123). Therefore, for this given example, washing the filter in 0.3 times SSC at 59.4-64.4 degrees C. will produce a stringency of hybridization equivalent to 90%; that is, DNA molecules with more than 10% sequence variation relative to the target BSMR cDNA will not hybridize. Alternatively, washing the hybridized filter in 0.3 times SSC at a temperature of 65.4-68.4 degrees C. will yield a hybridization stringency of 94%; that is, DNA molecules with more than 6% sequence variation relative to the target BSMR cDNA molecule will not hybridize. The above example is given entirely by way of theoretical illustration. One skilled in the art will appreciate that other hybridization techniques may be utilized and that variations in experimental conditions will necessitate alternative calculations for stringency.

In preferred embodiments of the present invention, stringent conditions may be defined as those under which DNA molecules with more than 25% sequence variation (also termed “mismatch”) will not hybridize. In a more preferred embodiment, stringent conditions are those under which DNA molecules with more than 15% mismatch will not hybridize, and more preferably still, stringent conditions are those under which DNA sequences with more than 10% mismatch will not hybridize. In a most preferred embodiment, stringent conditions are those under which DNA sequences with more than 6% mismatch will not hybridize.

The degeneracy of the genetic code further widens the scope of the present invention as it enables major variations in the nucleotide sequence of a DNA molecule while maintaining the amine acid sequence of the encoded protein. For example, the second amine acid residue of the BSMR protein is alanine. This is encoded in the BSMR cDNA by the nucleotide codon triplet GCT. Because of the degeneracy of the genetic code, three other nucleotide codon triplets—GCT, GCC and GCA—also code for alanine. Thus, the nucleotide sequence of the BSMR cDNA could be changed at this position to any of these three codons without affecting the amine acid composition of the encoded protein or the characteristics of the protein. The genetic code and variations in nucleotide codons for particular amine acids is presented in Tables 4 and 5. Based upon the degeneracy of the genetic code, variant DNA molecules may be derived from the cDNA molecules disclosed herein using standard DNA mutagenesis techniques as described above, or by synthesis of DNA sequences. DNA sequences which do not hybridize under stringent conditions to the cDNA sequences disclosed by virtue of sequence variation based on the degeneracy of the genetic code are herein T comprehended by this invention.

TABLE 3
The Genetic Code
First Third
Position
Second Position Position
(5′ end)

| T | C | A | G | (3′ end) |
|---|---|---|---|---|
| T | Phe | Ser | Tyr | Cys T |
| | Phe | Ser | Tyr | Cys C |
| | Leu | Ser | Stop (och) | |
| | | Stop A | | |
| | Leu | Ser | Stop (amb) | |
| | | Trp G | | |
| C | Leu | Pro | His | Arg T |
| | Leu | Pro | His | Arg C |
| | Leu | Pro | Gln | Arg A |
| | Leu | Pro | Gln | Arg G |
| A | Ile | Thr | Asn | Ser T |
| | Ile | Thr | Asn | Ser C |
| | Ile | Thr | Lys | Arg A |
| | Met | Thr | Lys | Arg G |
| G | Val | Ala | Asp | Gly T |
| | Val | Ala | Asp | Gly C |
| | Val | Ala | Glu | Gly A |
| | Val (Met) | Ala | Glu | Gly G |

“Stop (och)” stands for the ocre termination triplet, and “Stop (amb)” for the amber. ATG is the most common initiator codon; GTG usually codes for valine, but it can also code for methionine to initiate an mRNA chain.

TABLE 4
The Degeneracy of the Genetic Code

| Number of | | Total |
|---|---|---|
| Synonymous | | Number of |
| Codons | Amino Acid | Codons |
| 6 | Leu, Ser, Arg | 18 |
| 4 | Gly, Pro, Ala, Val, Thr | 20 |
| 3 | Ile 3 | |
| 2 | Phe, Tyr, Cys, His, Gln, | 18 |
| 1 | Glu, Asn, Asp, Lys | 2 |
| | Met,Trp | |

Total number of codons for amino acids 61
Number of codons for termination 3
Total number of codons in genetic code 64

One skilled in the art will recognize that the DNA mutagenesis techniques described above may be used not only to produce variant DNA molecules, but will also facilitate the production of proteins which differ in certain structural aspects from the BSMR protein, yet which proteins are clearly derivative of this protein and which maintain the essential characteristics of the BSMR protein. Newly derived proteins may also be selected in order to obtain variations on the characteristic of the BSMR protein, as will be more fully described below. Such derivatives include those with variations in amino acid sequence including minor deletions, additions and substitutions.

While the site for introducing an amino acid sequence variation is predetermined, the mutation per se need not be predetermined. For example, in order to optimize the performance of a mutation at a given site, random mutagenesis may be conducted at the target codon or region and the expressed protein variants screened for the optimal combination of desired activity. Techniques for making substitution mutations at predetermined sites in DNA having a known sequence as described above are well known.

Amino acid substitutions are typically of single residues; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. Obviously, the mutations that are made in the DNA encoding the protein must not place the sequence out of reading frame and preferably will not create complementary regions that could produce secondary mRNA structure (EP 75,444A).

Substitutional variants are those in which at least one residue in the amino acid sequence has been removed and a different residue inserted in its place. Such substitutions generally are made in accordance with the following Table 5 when it is desired to finely modulate the characteristics of the protein. Table 5 shows amino acids which may be substituted for an original amino acid in a protein and which are regarded as conservative substitutions.

TABLE 5

| Original | Conservative |
|---|---|
| Residue | Substitutions |
| Ala | ser |
| Arg | lys |
| Asn | gln, his |
| Asp | glu |
| Cys | ser |
| Gln | asn |
| Glu | asp |
| Gly | pro |
| His | asn; gln |
| Ile | leu, val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 5, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine.

The effects of these amino acid substitutions or deletions or additions may be assessed for derivatives of the BSMR protein by analyzing the ability of the derivative proteins to complement the sensitivity to DNA cross-linking agents exhibited by BSMR cells. These assays may be performed by transfecting DNA molecules encoding the derivative proteins into BSMR cells as described above.

The BSMR gene, BSMR cDNA, DNA molecules derived therefrom and the protein encoded by the cDNA and derivative DNA molecules may be utilized in aspects of both the study of BSMR and for diagnostic and therapeutic applications related to BSMR. Utilities of the present invention include, but are not limited to, those utilities described in the examples presented herein. Those skilled in the art will recognize that the utilities herein described are not limited to the specific experimental modes and materials presented and will appreciate the wider potential utility of this invention.

Having herein provided nucleotide sequences that code for BSMR and fragments of BSMR and having provided the essential BSMR biochemical-to-biological relationships for use of those sequences for drug discovery, intervention and diagnosis of bone strength and mineralization status, correspondingly provided are the complementary DNA strands of the cDNA molecule and DNA molecules which hybridize under stringent conditions to the BSMR cDNA molecule or its complementary strand. Such hybridizing molecules include DNA molecules differing only by minor sequence changes, including nucleotide substitutions, deletions and additions. Also comprehended by this invention are isolated oligonucleotides comprising at least a segment of the cDNA molecule or its complementary strand, such as oligonucleotides which may be employed as effective DNA hybridization probes or primers useful in the polymerase chain reaction. Hybridizing DNA molecules and variants on the BSMR cDNA may readily be created by standard molecular biology techniques.

The detection of specific DNA mutations may be achieved by methods such as hybridization using specific oligonucleotides (Wallace et al. (1986). Cold Spring Harbor Symp. Quant. Biol. 51:257-261), direct DNA sequencing (Church and Gilbert (1988). Proc. Natl. Acad. Sci. USA 81:1991-1995), the use of restriction enzymes (Flavell et al. (1978). Cell 15:25), discrimination on the basis of electrophoretic mobility in gels with denaturing reagent (Myers and Maniatis (1986). Cold Spring Harbor Symp. Quant. Biol. 51:275-284), RNase protection (Myers et al. (1985). Science 230:1242), chemical cleavage (Cotton et al. (1985). Proc. Natl. Acad. Sci. USA 85:4397-4401), and the ligase-mediated detection procedure (Landegren et al., 1988).

Oligonucleotides specific to normal or mutant sequences are chemically synthesized using commercially available machines, labelled radioactively with isotopes (such as .sup.32 P) or non-radioactively (with tags such as biotin (Ward and Langer et al. Proc. Natl. Acad. Sci. USA 78:6633-6657 1981), and hybridized to individual DNA samples immobilized on membranes or other solid supports by dot-blot or transfer from gels after electrophoresis. The presence or absence of these specific sequences are visualized by methods such as autoradiography or fluorometric or colorimetric reactions (Gebeyehu et al. Nucleic Acids Res. 15:4513-4534 1987).

Sequence differences between normal and mutant forms of that gene may also be revealed by the direct DNA sequencing method of Church and Gilbert (1988). Cloned DNA segments may be used as probes to detect specific DNA segments. The sensitivity of this method is greatly enhanced when combined with PCR (Stofiet et al. Science 239:491-494, 1988). In this approach, a sequencing primer which lies within the amplified sequence is used with double-stranded PCR product or single-stranded template generated by a modified PCR. The sequence determination is performed by conventional procedures with radiolabeled nucleotides or by automatic sequencing procedures with fluorescent tags.

Sequence alterations may occasionally generate fortuitous restriction enzyme recognition sites or may eliminate existing restriction sites. Changes in restriction sites are revealed by the use of appropriate enzyme digestion followed by conventional gel-blot hybridization (Southern (1975). J. Mol. Biol. 98:503). DNA fragments carrying the site (either normal or mutant) are detected by their reduction in size or increase of corresponding restriction fragment numbers. Genomic DNA samples may also be amplified by PCR prior to treatment with the appropriate restriction enzyme; fragments of different sizes are then visualized under UV light in the presence of ethidium bromide after gel electrophoresis.

Genetic testing based on DNA sequence differences may be achieved by detection of alteration in electrophoretic mobility of DNA fragments in gels with or without denaturing reagent. Small sequence deletions and insertions can be visualized by high-resolution gel electrophoresis. For example, a PCR product with small deletions is clearly distinguishable from a normal sequence on an 8% non-denaturing polyacrylamide gel (WO 91/10734, ) Nagamine et al. Am. J. Hum. Genet. 45:337-339 1989). DNA fragments of different sequence compositions may be distinguished on denaturing formamide gradient gels in which the mobilities of different DNA fragments are retarded in the gel at different positions according to their specific “partial-melting” temperatures (Myers et al. Science 230:1242 1985). Alternatively, a method of detecting a mutation comprising a single base substitution or other small change could be based on differential primer length in a PCR. For example, an invariant primer could be used in addition to a primer specific for a mutation. The PCR products of the normal and mutant genes can then be differentially detected in acrylamide gels.

In addition to conventional gel-electrophoresis and blot-hybridization methods, DNA fragments may also be visualized by methods where the individual DNA samples are not immobilized on membranes. The probe and target sequences may be both in solution, or the probe sequence may be immobilized (Saiki et al., Proc. Nat. Acad. Sci. USA 86:6230-6234 1989). A variety of detection methods, such as autoradiography involving radioisotopes, direct detection of radioactive decay (in the presence or absence of scintillant), spectrophotometry involving calorigenic reactions and fluorometry involved fluorogenic reactions, may be used to identify specific individual genotypes.

If more than one mutation is frequently encountered in the BSMR gene, a system capable of detecting such multiple mutations would be desirable. For example, a PCR with multiple, specific oligonucleotide primers and hybridization probes may be used to identify all possible mutations at the same time (Chamberlain et al. Nucl. Acids Res. 16:1141-1155 (1988)). The procedure may involve immobilized sequence-specific oligonucleotides probes (Saiki et al., Proc. Nat. Acad. Sci. USA 86:6230-6234 1989).

Use of BSMR Effectors and BSMR Expression for Ocular Development

In an embodiment of the invention a pharmaceutical agent alters the BSMR regulatory system to improve eye development and vascular growth in the eye. That is, effectors of BSMR function may be used to treat a range of eye disorders such as diabetic retinopathy, hypertensive retinopathy and retinopathy of prematurity, in which normal vascular growth and integrity of ocular vessels is disrupted. This embodiment arises from the discovery that the BSMR gene product is important to eye development. More specifically, it was discovered that a defect in the BSMR gene causes a defect in the normal involution of the primary vitreal vasculature that normally begins during the 13th gestational week. Failure of this process leads to fibrosis and contracture. Traction on other ocular structures by this fibrosing vitreal vasculature affects vision by creating retinal folds, retinal detachment, and in the extreme case, phthisis bulbi.

Without wishing to be bound by any one theory of this embodiment of the invention, it is believed that transient expression of LRP5 by cells within the vitreal vasculature, or by other vitreous constituents, is responsible for this involution process. Two other Mendelian genetic ocular disorders, autosomal dominant familial exudative vitreoretinopathy (FEVR) and autosomal dominant neovascular inflammatory retinopathy (VRNI) also map to an interval containing BSMR (LRP5). FEVR appears to result from impaired growth of the peripheral retinal vasculature, with resultant formation of fibrovascular vitreal membranes that lead to retinal traction and detachment. VRNI appears to result from an inflammatory process with occlusion of peripheral retinal vasculature and subsequent new vessel growth. Visual loss is a common feature in both syndromes, although there is variable expression and obligate carriers can be asymptomatic. In an embodiment of the invention, such visual loss is prevented or ameliorated by administering a BSMR effector or another technique such as described herein for altering bone mineralization.

Detection of Bone Status by Assay of BSMR

In an embodiment of the invention an individual's bone status disposition is detected by quantitating the level of BSMR protein fragments in the cells, in the blood or in other extracellular fluid of an individual. Such measurements are useful for detecting reduced levels of the BSMR protein which result from, for example, mutations in the promoter regions or the leader segment of the BSMR gene or from altered processing of full length BSMR protein. The determination of reduced BSMR protein levels is an alternative or supplement to the direct determination of BSMR by nucleotide sequence determination outlined above. The availability of antibodies specific to the BSMR protein allows the quantitation of cellular BSMR protein by immunoassay methods which are well known in the art and are presented in Harlow and Lane, ANTIBODIES, A LABORATORY MANUAL, Cold Spring Harbor Laboratory, New York (1988).

For the purposes of quantitating the BSMR protein and or for examining for the presence of epitopes on the BSMR protein, a biological sample of the subject is required. Such a biological sample may be obtained from body fluids such as those present in peripheral blood, urine, saliva, tissue biopsy, amniocentesis samples, surgical specimens and autopsy material. Immunoassay quantitation of BSMR protein, or of particular epitopes of BSMR protein and comparison with normal levels of other protein or epitopes can provide information about the expression or processing of BSMR protein. A significant (preferably 50% or greater) reduction in the amount of BSMR protein (or a particular epitope) in the cells of a subject compared to a control measurement would indicate that the subject may be an OPS carrier or has poor bone strength and mineralization status.

In a preferred embodiment, antibodies are used that discriminate between regions of the BSMR protein, for example between the distal (away from the membrane surface) amino terminal portion and the ligand binding region portion that is close to the cell membrane exterior surface. In this embodiment a shortened BSMR derivative may be determined by differential antibody binding.

Monoclonal and/or polyclonal antibodies may be produced to normal BSMR protein and to mutant forms of this protein. Optimally, antibodies raised against the BSMR protein would specifically detect the BSMR protein. That is, such antibodies would recognize and bind the BSMR protein and would not substantially recognize or bind to other proteins found in human cells. The determination that an antibody specifically detects the BSMR protein is made by any one of a number of standard immunoassay methods; for instance, the Western blotting technique (Sambrook et al. (1989). IN MOLECULAR CLONING: A LABORATORY MANUAL, Cold Spring Harbor, N.Y.). To determine that a given antibody preparation (such as one produced in a mouse) specifically detects the BSMR protein by Western blotting, total cellular protein is extracted from human cells (for example, lymphocytes) and electrophoresed on a sodium dodecyl sulfate-polyacrylamide gel. The proteins are then transferred to a membrane (for example, nitrocellulose) by Western blotting, and the antibody preparation is incubated with the membrane. After washing the membrane to remove non-specifically bound antibodies, the presence of specifically bound antibodies is detected by the use of an anti-mouse antibody conjugated to an enzyme such as alkaline phosphatase; application of the substrate 5—bromo—4—chloro—3—indolyl phosphate/nitro blue tetrazolium results in the production of a dense blue compound by immuno-localized alkaline phosphatase.

Antibodies which specifically detect the BSMR protein will, by this technique, be shown to bind to the BSMR protein band (which will be localized at a given position on the gel determined by its molecular weight). Non-specific binding of the antibody to other proteins may occur and may be detectable as a weak signal on the Western blot. The non-specific nature of this binding will be recognized by one skilled in the art by the weak signal obtained on the Western blot relative to the strong primary signal arising from the specific antibody—BSMR protein binding.

In a preferred embodiment, antibody binding is used to detect the presence of morphogenic forms of BSMR. For example, a monoclonal antibody directed against a particular ligand binding site region of the BSMR protein can be used to detect differences in that binding site between individuals.

The principal embodiments having been reviewed, examples of selected embodiments are next presented. The examples are not meant to limit the meaning of the claimed invention in any way but merely represent selected features of some of the embodiments.

### EXAMPLE 1

In this example, tissue samples were obtained from 7 OPS patients. DNAs from the samples were examined for the presence of mutations in various proteins. One of the protein genes, (LRP5) showed drastic DNA changes as summarized in Table 1.

TABLE 1

| | Genetic Alteration (numbers start with |
|---|---|
| OPS Patient | the first translated base pair) |
| 1 | C1282T (creates a stop codon) |
| 2 | G2202A (creates a stop codon) |
| 3 | 1467delG (creates a frame-shift) |
| 4 | G1453T (creates a stop codon) |
| 5 | 2150insT (creates a frame-shift) |
| 6 | C2557T (creates a stop) |
| 7 | 3804delA (creates a frame-shift) |

These results indicate that OPS is linked to a severe alteration in a single protein coded for by a gene that has been known by various workers as LRP5, Lrp7, and Lr3, as for example described in Hey et al., Gene 216: 103-111 (1998), Chen et al., Genomics 55: 314-321 (1999), Dong et al., Biochem. Biophys. Res. Commun. 251: 784-790 (1998) and Hey et al., Gene 216: 103-111 (1998).

### EXAMPLE 2

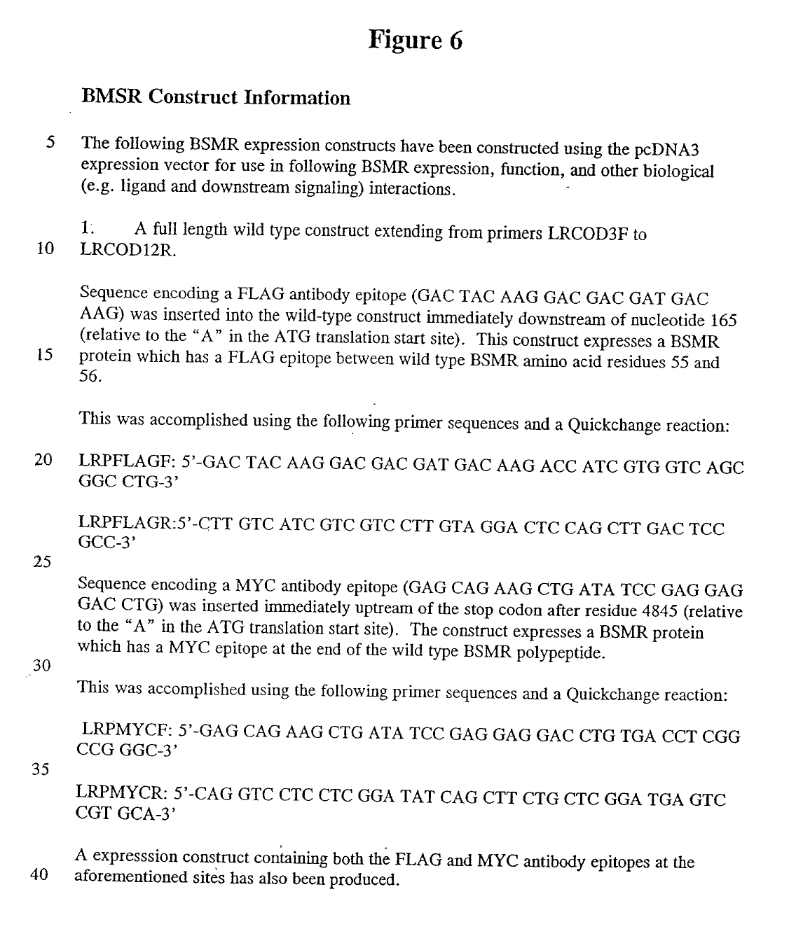

In this example, BSMR expression constructs are constructed using the pcDNA3 expression vector and are used for detecting BSMR expression, function, ligand interactions and downstream signaling interactions. The amino acid sequences of the constructs are listed in FIG. 6.

Using standard techniques, a full length wild type construct extending from primers LRCOD3F to LRCOD12R was constructed. A sequence encoding a FLAG antibody epitope (GAC TAC AAG GAC GAC GAT GAC AAG) (SEQ ID NO:84) was inserted into the wild-type construct immediately downstream of nucleotide 165 (relative to the “A” in the ATG translation start site). This construct expressed a BSMR protein having a FLAG epitope between wild type BSMR amino acid residues 55 and 56. The construct was prepared by the following primer sequences and a Quickchange reaction:

| | |
|---|---|
| LRPFLAGF: 5′-GAC TAC AAG GAC GAC GAT GAC AAG ACC ATC GTG GTC AGC GGC CTG-3′ | (SEQ ID NO:85) |
| LRPFLAGR: 5′-CTT GTC ATC GTC GTC CTT GTA GGA CTC CAG CTT GAC TCC GCC-3′ | (SEQ ID NO:86). |

Using standard techniques, a construct was made by inserting a sequence encoding an MYC antibody epitope (GAG CAG AAG CTG ATA TCC GAG GAG GAC CTG) (SEQ ID NO:87) immediately uptream of the stop codon after residue 4845 (relative to the “A” in the ATG translation start site). The construct expressed a BSMR protein having a MYC epitope at the end of the wild type BSMR polypeptide. This construct was constructed using the following primer sequences and a Quickchange reaction:

| | |
|---|---|
| LRPMYCF: 5′-GAG CAG AAG CTG ATA TCC GAG GAG GAC CTG TGA CCT CGG CCG GGC-3′ | (SEQ ID NO:88) |
| LRPMYCR: 5′-CAG GTC CTC CTC GGA TAT CAG CTT CTG CTC GGA TGA GTC CGT GCA-3′ | (SEQ ID NO:89). |

Using standard techniques, an expresssion construct containing both the FLAG and MYC antibody epitopes as described above at the aforementioned sites was produced.

### EXAMPLE 3

In this example, functional BSMR protein, useful for discovering pharmaceuticals useful for regulating bone strength and mineralization were constructed having functional Fc and myc epitopes, using standard construction techniques. More information about the amino acid sequences of the constructs is provided in FIG. 6.

An in-frame Hind III restriction enzyme site was created in a first full length wild-type BSMR construct, at the amino-terninus of the transmembrane domain, at nucleotide residue 4248. Separately, an in-frame Hind III restriction enzyme site was created in a second full length wild-type BSMR construct at the carboxy terminus. Using the Hind III restriction sites in the first construct, a Hind III/Not I fragment of the human IgG1 Fc domain (GenBank accession X70421.1) was inserted in-frame to create a functional Fc epitope at the carboxy-end of the BSMR extracellular domain.

Using the Hind III restriction sites in the second construct, a Hind III/Not I fragment of the human IgG1 Fc domain (GenBank accession X70421.1) was inserted in-frame to create a functional Fc epitope at the carboxy-end of the full length BSMR protein. Separately, the second construct also was also cloned into the pcDNA3.1/myc—His B vector (Invitrogen) and expressed a full length myc-tagged BSMR protein. This latter expressed protein differs from the protein expressed from the first modified construct in the way that the myc epitope was added to the BSMR protein. Separately, the second construct also was cloned into the pEGFR—NI vector (Clontech) to generate a full length BSMR protein with green fluorescent protein added in-frame at the carboxy-terminus.

These representative constructs are used to make altered protein having desired labels and epitopes for use in drug discovery and in diagnosis of bone strength and mineralization.

### EXAMPLE 4

In this example conditioned media containing the dominant-negative secreted form of BSMR reduced alkaline phosphatase expression in primary calvarial osteoblast cultures and reduced bone formation in calvarial explant cultures. Calvarial cells exposed to Wnt3a conditioned media increased their alkaline phosphatase activity compared to controls. Cells exposed to conditioned media from cells expressing wild type BSMR did not differ from controls. Cells exposed to conditioned media from cells expressing the secreted BSMRdeltaTM form of BSMR had reduced alkaline phosphatase activity compared to controls, indicating that the decoy BSMR receptor had interfered with osteoblast function. In another study, explants that were cultured in the presence of conditioned media from cells expressing BSMRdeltaTM consistently had less bone formation, as detetermined by von Kossa staining, than did explants cultured in conditioned media from cells expressing wild-type BSMR. This BSMR is able to bind extracellular growth factors, such as Wnts, that are important during bone growth.

### EXAMPLE 5

This example demonstrates tissue specific expression of BSMR activity by in situ hybridization.

Calvaria were dissected from E19 or newborn Swiss mice. Calvaria were cleaned of extraneous tissue. For primary calvarial osteoblast isolation, the calvaria were minced, and digested with Trypsin and Collagenase P for 20 minutes at 37 C. Recovered cells were plated at a density of 2.5×10⁴ cells per well in 24 well culture dishes and cultured in MEM containing 10% FBS and antibiotic/antimycotic. Following 48 hours in culture, the cells were switched to conditioned media that was supplemented with ascorbic acid and (α—glycerolphosphate. Media was changed every two days and cells were harvested for alkaline phosphatase activity and DNA quantitation after one week.

For calvarial explant cultures, calveria were embedded in 0.2 ml of sterile low melt agarose containing DMEM, 10% FBS, and antibiotics. The embedded calvaria were then overlayed with an additional 1 ml of culture media. Following 24 hours in culture the media was switched to conditioned that was supplemented with ascorbic acid and α—glycerolphosphate Media was changed every two days and explants were harvested for histologic analysis after 5 days. von Kossa staining was performed using standard methods.

C57BL/6 wild type mouse embryos were fixed in 4% paraformaldehyde in 0.1 M phosphate buffer (PB), pH 7.4 at 4° C. overnight. After immersion in 20% sucrose in PB, tissues were embedded in OCT compound in an ethanol bath with dry ice and stored at −80° C. until used. Frozen tissue sections were cut at a thickness of 8 μm, mounted onto glass slides (Fisher, Superfrost/Plus slides) and allowed to dry. The sections were treated with 0.2N HC1 for 15 minutes, washed twice in PBS, and incubated with proteinase K (Sigma; 10, μg/ml in 10 mM Tris—HC1, pH 7.4, 1 mM CaC1₂) at 37° C. for 10 minutes. After washing twice with PBS, tissues were briefly post-fixed with 4% paraformaldehyde in PB, pH 7.4, followed by acetylation with acetic anhydride in 0.1 M triethanolamine solution for 15 minutes at room temperature. The sections were washed twice with PBS, dehydrated through a graded alcohol series and allowed to dry in air.

BSMR specific antisense probes were synthesized to cover 700 basepairs of cDNA. Digoxigenin—11—UTP labeled single strand ribo-probes were prepared using the DIG RNA labeling kit (Roche) by in vitro transcription according to the manufacturer's instructions. The ribo-probes were denatured at 85° C. for 3 minutes, and hybridization was carried out overnight with the probe (approximate concentration was 100 ng/ml) in 50% formamide, 10 mM Tris pH 7.6, 600 mM NaC1, 0.25% SDS, 10% dextran sulphate, 1×Denhardt's, 10 mg/ml yeast total RNA, 1 mM EDTA pH 8.0 at 55° C. Excess probe and hybridization solution was rinsed off with 5×SSC at 56° C., and the tissues were soaked with 50% formamide in 2×SSC at 55° C. for 30 minutes. The tissues were treated with 10 μg/ml RNase A (Sigma) in RNase A buffer (0.5 M NaC1, 10 mM Tris pH 7.6, 1 mM EDTA) at 37° C. for 30 minutes, followed by a 20 minute wash in 2×SSC, 0.1% SDS, and two 20 minute washes in 0.2×SSC, 0.1% SDS at 52° C. The tissues were immersed in 1.5% blocking reagent for 30 minutes at room temperature, washed with DIGI buffer (100 mM Tris—HC1, 150 mM NaC1) and incubated with anti-digoxigenin antibody conjugated with alkaline phosphatase (Roche, 1:1000 dilution with DIGI buffer) for 1 hour at room temperature. After washing twice with DIGI buffer for 15 minutes and neutralizing in DIG3 buffer (100 mM Tris—HC1, pH 9.5, 100 mM NaC1, 50 mM MgC12) for 5 minutes, hybridization was detected using 4—nitroblue tetrazolium and 5—bromo—4—chloro—3—indyl—phosphate as substrates for alkaline phosphatase. Incubation with substrate was carried out a 4° C. for 8 hours and the reaction was stopped with 10 mM Tris—HC1 pH 7.6 and 1 mM EDTA. After counter staining with 0.2% methyl-green, slides were allowed to dry and mounted with Glycergel (DACO). The specimens were observed and recorded by a SPOT camera (Diagnostic Instruments Inc.).

### EXAMPLE 6

This example demonstrates the use of various effectors including BMP2, Wnt3a, Wnt1, beta—catenin, TCF—1, and Gal4 on BSMR linked bone metabolism activity.

BMP2 Induction of ST2 Cell Osteoblastic Differentiation

Cells of the murine bone marrow stromal cell line ST2 was cultured in RPMI 1640 (Cellgro) supplemented with 10% FBS, 50 U/ml penicillin and 50 ug/ml streptomycin (GIBCO-BRL). Cells were plated at a density of 2×10^{4/}cm² in 25 cm² flasks and, when confluent, osteoblastic differentiation was induced by the addition of recombinant human BMP2 (Aventis) at 300 ng/ml. Cell cultures were then continued for up to 144 hours. Total RNA from cells was harvested at 0, 0.5, 1, 2, 3, 4, and 6 days following BMP2 addition using the Trizol reagent (GIBCO-BRL) following the manufacturer's recommendation.

Northern Blot Analysis

Total RNA isolated from cultured cells was separated in 1% agarose-formaldehyde gels (5 μg total RNA/lane) and transferred to Hybond—N membrane (Amersham Pharmacia) using standard methods. Replicate Northern blots were made to minimize potential differences in observed RNA expression associated with repeat hybridization. Equal mRNA loading across gels and transfer to membrane were determined by ethidium bromide staining. Hybridization was performed overnight using Ultrahyb (Ambion Inc.) following the manufacturer's recommendation. cDNA probes were random prime labeled using standard methods. Analyzed mouse probes included Lrp5: nt 4101-5092 (Genbank accession # NM_{—}008513), Lrp6: nt 2918-3769 (Genbank accession # NM_{—}008514), ALP, BGP, Collal, (Sabatakos et al., 2000) and Actb.

BSMR Expression Constructs and Stable BSMR Expressing Cell Lines

BSMR expression constructs were constructed using the pcDNA3 expression vector (Invitrogen). Full length cDNA was amplified by RT-PCR from human fibroblasts and subcloned. A FLAG antibody epitope (GAC TAC AAG GAC GAC GAT GAC AAG) was inserted into the wild-type construct immediately downstream of nucleotide 165 (the insertion site is relative to the “A” in the ATG translation start site) using QUIKChange (Stratagene). QUIKChange was also used to create expression constructs containing the 1270fsl438stop and Gln853Stop disease-causing mutations. An endogenous Pml I restriction site, downstream of the transmembrane domain, was used to generate the BSMRdeltaC dominant negative receptor by truncating the protein and removing the intra-cytoplasmic tail. An endogenous Apa I restriction site at the beginning of the transmembrane domain was used to generate the BSMRdeltaTM dominant negative receptor that lacks the transmembrane domain and cytoplasmic tail. The entire BSMR coding sequences of all constructs were confirmed by sequencing. Stable ST2 cell lines expressing either wild type or a second LRP5□C' construct, which was generated using an endogenous BsrG I restriction enzyme site and lacks the final 100 amino residues of the cytoplasmic tail, were created picking individual colonies following selection with G418.

Wnt, Constitutive-active β—catenin, TCF—1, and TCF—luciferase, Gal4—Smad, and Gal4—luciferase Constructs

Mouse TCF—1 was amplified by RT—PCR and the nucleotide sequence was confirmed by DNA sequence analysis and cloned into pcDNA3.1 vector (Invitrogen). The TCF—luciferase reporter construct, TOPflash, was obtained from Upstate Biotechnology.

Wnt and BSMR Responsiveness Using C3H10T1/2, ST2, and COS—7 Cell Lines

Cell lines were cultured (5% CO₂ at 37° C.) respectively in □—MEM, RPMI and DMEM medium supplemented with 10% of heat inactivated fetal calf serum. ST—2 stable cell lines were maintained in the corresponding culture medium and supplemented with G418 (500 μg/ml). For treatment or transient transfection, cells were plated at 2×10⁴/cm² and 24 h later treatment or transfections were carried out as indicated below. Cells plated in 24-well plates were transiently transfected with the indicated construct (1 microgram) using DNA-lipid complex Fugene 6 (Boehringer Maheim) according to the manufacture's protocol. For luciferase reporter assays 20 ng of pRL-TK (Promega, Madison, WI), which encodes a Renilla luciferase gene downstream of a minimal HSV-TK promoter, was systematically added to the transfection mix to assess transfection efficienty. When required, controls were carried out by replacing constructs with corresponding empty vectors.

Sixteen hours after transfection, cells were washed, cultured in medium at 2% fetal calf serum and either left unstimulated or stimulated with BMP-2 (100 ng/ml) for additional 48 h. Luciferase or alkaline phosphatase activity was determined in cell lysates. When luciferase reporter constructs were used, luciferase assays were performed with the Dual Luciferase Assay Kit (Promega) according to the manufacturer's instructions. Ten μl of cell lysate was assayed first for firefly luciferase and then for Renilla luciferase activity. Firefly luciferase activity was normalized to Renilla luciferase activity. Alkaline phosphatase activity was determined in cell lysates using the Alkaline Phosphatase Opt kit (Roche Molecular Biochemicals). Cell lysates were analyzed for protein content using the micro—BCA Assay kit (Pierce), and alkaline phosphatase activity was normalized for total protein concentration. Each experiment was performed in triplicate, and was repeated three times. Data (plus and minus one standard deviation) from one representative experiment are presented in FIG. 11.

Generation of Wnt and BSMR Conditioned Media

Wnt3a and Wnt1 expressing cell lines were prepared as described above. Cells were maintained in DMEM containing 10% FBS and antibiotic/antimycotic. Ten ml of Wnt3a or Wnt1 containing culture media were collected every two days from 100 mm diameter culture dishes after cells became confluent.

BSMR conditioned media was generated by transiently transfecting 80% confluent COS—7 cells in 100 mm diameter dishes with 5μg plasmid DNA using Fugene 6 following the manufacturer's protocol. 10 ml each of conditioned medium was collected 24 and 48 hours after the cells were allowed to recover from the transfection. The presence of the protein in either the conditioned media or cell extract was confirmed by Western blotting with the anti-flag antibody.

### EXAMPLE 7

This example shows transcriptional regulation of frizzled receptors 1 and 2 and SFRPs 1,2 and 4 in the time-course of osteoblastic maturation of primary calvaria cells.

Murine calvaria cells were obtained from neonatal mice 1-2 days after birth by sequential collagenase digestion. Calvariae were removed and incubated at 37° C. in DMEM containing trypsin (0.5 mg/ml) and EDTA (1.5 mg/ml) under continuous agitation. Trypsin digests were discarded at 15 minutes and replaced with DMEM that contained 1 mg/ml of collagenase. cThe collagenase digests were discarded at 20 minutes and replaced with fresh enzyme dilution. The cells released between 20-40 minutes were collected by a short passive sedimentation step, and two centrifugation steps (400g, 10 minutes), The collected cells were cultured in proliferation medium (DMEM supplemented with 20% FCS and 2 mM glutamine) at a density of 2.5×10⁴ cells per cm² in Petri dishes (100 mm diameter). Calvaria cells were cultured until 80% confluence (time 0) and proliferation medium was replaced by differentiation medium (DMEM containing 10% FCS, 2 mM glutamine, 50 mg/ml ascorbic acid and 10 mM b—glycerolphosphate). Total RNAs were extracted with an RNAplus kit from Quantum, from cells harvested from culture at days 0, 2, 7,14, and 21. Changes in relative gene expression were assessed by using GeneChips (Affymetrix). Results (see FIG. 7) are expressed in ratios using the time 0 as denominator, significant changes in expression (pval<0.1 and ratios >1.5×) are indicated with **.

### EXAMPLE 8

This example shows the transcriptional regulation of Wnt's, frizzled receptors and SFRPs during maturation of human bone marrow (BMSC) and purified trabecular bone (NHBC) primary cells.

Prior to sorting, BMSC were seeded into culture flasks at a density of 2×10⁴/cm², and cultured in DMEM supplemented with 15% FBS and 100μM ascorbate—2—phosphate for 14 days. The cells were then redistributed in situ following treatment with collagenase and trypsin—EDTA, and cultured until a confluent monolayer had formed (2-3 weeks). Cultures of human bone-derived cells (NHBC) were obtained by seeding small fragments of trabecular bone in tissue culture flasks with DMEM supplemented with 10% FBS and 100 μM ascorbate—2—phosphate. Cultures were grown until a confluent monolayer had formed (5-6 weeks) and the cells removed from the trabecular chip following treatment with collagenase and trypsin—EDTA, and re-seeded into culture flasks and grown under identical conditions until confluent again.

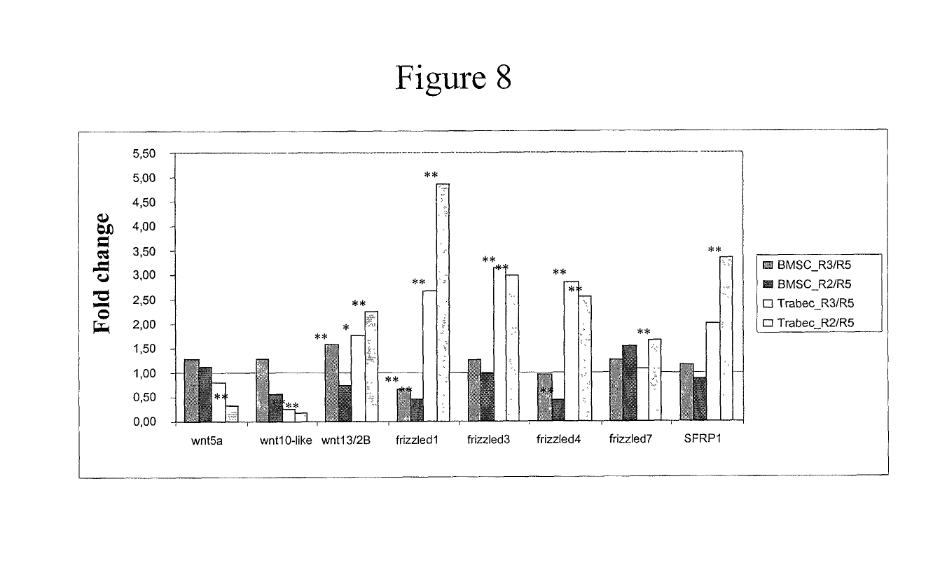

Both cell populations were harvested and dual labelled for STRO—1 and alkaline phosphatase as described by Stewart, K et al, JBMR 11:P 208 (1996) prior to sorting by flow cytometry. Sort regions were the set within each of the quadrants and cells were sorted into four populations. Cells recovered were re-analyzed by flow cytometry for purity, counted, then pelleted and stored at −80° C. The STRO—1+ fraction corresponded to less differentiated osteoblast precussors (R5), the STRO—1+/AP+ fraction corresponded to more mature osteoblasts (R3) and the AP+ fraction corresponded to mature osteoblasts (R2). Total RNAs were extracted with a RNAplus kit provided by Quantum. Changes in relative gene expression were assessed by GeneChips ™ from Affymetrix. Results (see FIG. 8) are expressed in ratios with values at time 0 as denominators. Significant changes in expression (pval<0.1 and ratios >1.5×) are indicated with **. Ratios greater than 1.5 and with pval<0.15 are indicated with *.

### EXAMPLE 9

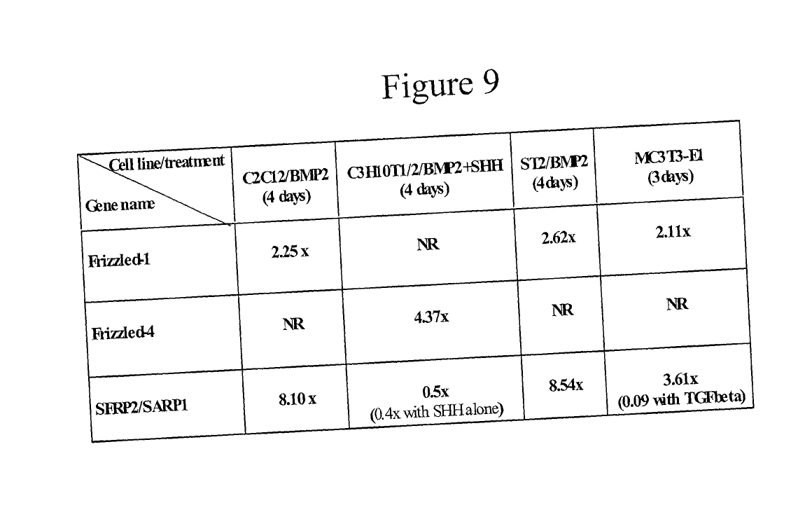

This example shows transcriptional regulation of frizzled receptors (1 and 4) and SFRP2 observed in mouse pluripotent and osteoblast-like cell lines. The cells described as above were treated as indicated with BMP2, BMP/SHH or TGFbeta and changes in relative gene expression were assessed using GeneChips ™ by Affymetrix. Results (see FIG. 9) are expressed in ratios using the expression values of the untreated cells in the denominator. Only the significant changes in expression (pval<0.1 and ratios >1.5 X) are shown.

### EXAMPLE 10

This example demonstrates induction of ALP activity (indicating bone formation) in C3H10T1/2 and ST2 cells by Wnt proteins and by β—catenin. All experiments were repeated three times and with triplicate samples. The data (showing plus and minus one standard deviation) from representative experiments are shown in the figures. Each of these BSMR effectors stimulated bone growth, as determined by ALP activity.

C3H10T1/2 and ST2 cells transiently were transfected with Wnt3a and with Wnt5a expression vectors. As a control, the empty vector pcDNA3 was used. ALP activity was measured in cell lysates prepared 72 h after transfection. The ALP activity values were normalized for protein content and expressed as nmol pnpp/min/μg of protein. FIG. 10a shows the results obtained with C3H10T1/2 cells and FIG. 10b shows the results obtained with ST2 cells.

In another experiment C3H10T1/2 cells transiently were transfected with either vector wild-type β—catenin (β—catenin) expressing vector or a stable mutant β—catenin (delta β—catenin*) expressing vector. Control cells were transfected with pcDNA3. ALP activity was measured in cell lysates prepared 72 h after transfection. FIG. 10c shows the results obtained. The ALP activity values shown in the figure were normalized for protein content and expressed as nmol pnpp/min/μg of protein.

### EXAMPLE 11

BSMR protein lacking a ligand binding region inhibits ALP activity induced by Wnt3a and BMP—2. COS cells were transiently transfected with TCF—1 expression construct, TOPflash, pTK—Renilla, and Wnt3a. As indicated below, either BSMR, BSMRdeltaC or BSMRdeltaTM were added to the transfection mix. As a control the empty vector pcDNA3 was used for transfection. Luciferase activity was determined in cell lysates 24 hours after transfection and normalized to renilla signals. Experiments were performed in triplicate in three independent experiments. The data (plus and minus one standard deviation) for a representative experiment are presented as luciferase fold induction compared to control in FIG. 11a. In a parallel experiment C3H10T1/2 and ST2 cells were transiently transfected with Wnt3a expression vector in the presence and absense of BSMR, BSMRdeltaC and BSMRdeltaTM expression constructs. ALP activities were measured in cell lysates 72 hours after transfection and the values were normalized to protein level. The results are shown in FIG. 11b.

In another parallel experiment ST2 cells were transiently transfected with either empty plasmid (pcDNA3), BSMRdeltaC expression plasmid or BSMRdeltaTM expression plasmid. Sixteen hours after transfection the cells were either left untreated (BMP—2-)or treated with BMP—2 at 100 ng/ml (BMP—2+) and cultured for an additional 72 hours. ALP activities were measured in cell lysates and normalized to protein level. ALP activities are expressed as nmol pnpp/min/μg of protein. Data (plus and minus one standard deviation) from one representative experiment are shown in FIG. 11c. All experiments were repeated three times and performed with triplicate samples.

### EXAMPLE 12

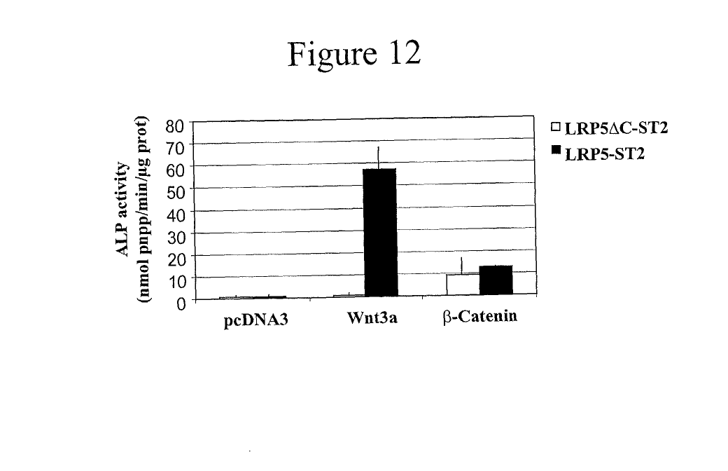

Wnt3a is a strong effector of BSMR protein and requires intact protein as ST—2 cells stably expressing BSMRdeltaC are not able to express ALP in response to Wnt3a. β—catenin is an effector of bone synthesis. ST2 cells stably expressing either BSMRdeltaC (BSMRdeltaC—ST2) or BSMR (BSMR—ST2) were transfected transiently with Wnt3a or control empty vector. ALP activities were determined in cell lysates 72 hours after transfection and normalized to protein levels. ALP activities are expressed as nmol pnpp/min/μg of protein and shown in FIG. 12. All experiments were repeated three times and performed in triplicate. Data (plus and minus one standard deviation) from one representative experiment are presented.

### EXAMPLE 13

This example demonstrates the effect of BSMR on regulation of angiogenesis in aortic explant cultures.

Thoracic aortas were excised from 4- to 7-month-old mice, dissected carefully to clean off surrounding fibroadipose tissue, rinsed extensively with phosphate-buffered saline, sectioned into approximately 1 mm rectangular pieces, and embedded in a type I collagen gel. A collagen solution was obtained by mixing 7 volumes of 4.15 mg/ml collagen ( Becton Dickinson, Bedford, Mass.) with 2 volumes of 1.17% NaHCO₃ and 1 volume of 10× MEM. From this solution a gel was made by adding 250 ul chilled collagen solution into pre-chilled 24 well culture plates on ice and geled at 37° C. After collagen gelation, 0.5 ml of medium containing 0.25 ml of SFM—Endothelial medium (Life Technologies Inc. Rockville, Md.) and 0.25 ml of conditioned medium from COS—7 cells expressing either full-length BSMR (control 1) or a soluble, secreted, decoy form of BSMR (BSMRdeltaTM), was added and changed every other day. An additional control (control 2) consisted of SFM—Endothelial medium only. To some of the cultures conditioned medium containing the BSMR ligand Wnt3a was also added. The amount of Wnt3a added was estimated to represent 10% of the amount of soluble BSMRdeltaTM decoy receptor.

The aortic explants were cultured at 37° C., 5% CO2 for 11 days. The cultures were fixed in 4% paraformaldehyde in phosphate-buffered saline for 1 hour and stained with anti-CD31 antibodies to visualize microvessel outgrowths. Microscopical examination showed that Wnt3a stimulated and BSMRdeltaTM reduced angiogenesis in this assay. The results indicate that BSMR and molecules that stimulate or inhibit the activity of BSMR may be used to control angiogenesis in diseases where abnormal angiogenesis occurs, such as in tumors, diabetic retinopathy, exudative vitreoretinopathy (both dominant and recessive forms).

Each cited publication is incorporated in its entirety by reference. Priority documents 60/304,851; 60/226,119 and 60/234,337 are incorporated in their entireties by reference.

One skilled in the art can modify the teachings presented herein in arrangement and detail without departing from the principles taught. We claim all modifications coming within the spirit and scope of the claims presented below.

We claim:

1. An isolated DNA molecule useful for the diagnosis or therapy of osteoporosis, selected from the group consisting of:
(a) a DNA molecule having a nucleotide sequence encoding an amino acid sequence as shown in FIG. 2;
(b) a DNA molecule capable of specific hybridization under stringent conditions to a DNA molecule according to (a), and which encodes a protein that, when introduced into human osteoblasts, increases the ability of the osteoblasts to synthesize bone;
(c) a DNA molecule having a nucleotide sequence which is degenerate as a result of the genetic code to the encoded protein amino acid sequence according to (b) and which encodes a protein that, when introduced into osteoblast cells of humans, increases the ability of the cells to synthesize bone;
(d) a DNA molecule capable of specific hybridization under stringent conditions to a DNA molecule according to (a), and which encodes a protein that, when introduced into endothelial cells of humans, increases the ability of the endothelial cells to form vascular tissue; and
(e) a DNA molecule having a nucleotide sequence which is degenerate as a result of the genetic code to the encoded protein amino acid sequence according to (d), and which encodes a protein that, when introduced into cells of humans, increases the ability of the cells to form vascular tissue.

2. A nucleic acid used for a method of determining predisposition to osteoporosis of a human subject by incubating the nucleic acid with nucleic acid sequences derived from a sample from the subject, the nucleic acid consisting of at least 10 consecutive nucleotides of a DNA sequence selected from the sequence shown in FIG. 2 or a complementary strand of said sequence.

3. A nucleic acid as described in claim 2, wherein the method of incubating the nucleic acid with nucleic acid sequences derived from a sample of the subject utilizes incubation conditions of 750 mM NaC1, 50 mM NaPhosphate, 5 mM EDTA, pH 7.4 and a temperature of 25-30 degrees C.

4. A nucleic acid used for a method of determining predisposition to osteoporosis of a human subject according to claim 3 wherein the nucleic acid consists of at least 25 consecutive nucleotides of a DNA sequence.

5. An expression vector comprising a promoter that is operably linked with a DNA molecule useful for the diagnosis or therapy of osteoporosis, the DNA molecule selected from the group consisting of:
(a) a DNA molecule having a nucleotide sequence encoding an amino acid sequence as shown in FIG. 2;
(b) a DNA molecule capable of specific hybridization under stringent conditions to a DNA molecule according to (a), and which encodes a protein that, when introduced into human osteoblasts, increases the ability of the osteoblasts to synthesize bone;
(c) a DNA molecule having a nucleotide sequence which is degenerate as a result of the genetic code to the encoded protein amino acid sequence according to (b) and which encodes a protein that, when introduced into osteoblast cells of humans, increases the ability of the cells to synthesize bone;
(d) a DNA molecule capable of specific hybridization under stringent conditions to a DNA molecule according to (a), and which encodes a protein that, when introduced into endothelial cells of humans, increases the ability of the endothelial cells to form vascular tissue; and
(e) a DNA molecule having a nucleotide sequence which is degenerate as a result of the genetic code to the encoded protein amino acid sequence according to (d), and which DNA molecule encodes a protein that, when introduced into endothelial cells of humans, increases the ability of the endothelial cells to form vascular tissue, wherein the expression vector codes for a transmembrane protein that modulates bone density.

6. A method for modulating bone density, the method comprising the step of introducing an expression vector into a bone cell, wherein the expression vector comprises a promoter that is operably linked with a DNA molecule useful for the diagnosis or therapy of osteoporosis, the DNA molecule selected from the group consisting of:
(a) a DNA molecule having a nucleotide sequence encoding an amino acid sequence as shown in FIG. 2;
(b) a DNA molecule capable of specific hybridization under stringent conditions to a DNA molecule according to (a), and which encodes a protein that, when introduced into human osteoblasts, increases the ability of the osteoblasts to synthesize bone;
(c) a DNA molecule having a nucleotide sequence which is degenerate as a result of the genetic code to the encoded protein amino acid sequence according to (b) and which encodes a protein that, when introduced into osteoblast cells of humans, increases the ability of the cells to synthesize bone;
(d) a DNA molecule capable of specific hybridization under stringent conditions to a DNA molecule according to (a), and which encodes a protein that, when introduced into endothelial cells of humans, increases the ability of the endothelial cells to form vascular tissue; and
(e) a DNA molecule having a nucleotide sequence which is degenerate as a result of the genetic code to the encoded protein amino acid sequence according to (d), and which DNA molecule encodes a protein that, when introduced into endothelial cells of humans, increases the ability of the endothelial cells to form vascular tissue.

7. A method of using an expression vector to select a test ligand that modulates bone density, comprising the steps of:
(a) introducing the expression vector into a host cell to produce a recombinant host cell that expresses the receptor on its surface;
(b) culturing the recombinant host cell; and
(c) assaying binding either directly or indirectly between the expressed receptor from the cultured recombinant host cell and the test ligand, wherein the expression vector comprises a promoter that is operably linked with a DNA molecule useful for the diagnosis or therapy of osteoporosis, the DNA molecule selected from the group consisting of:
(i) a DNA molecule having a nucleotide sequence encoding an amino acid sequence as shown in FIG. 2;
(ii) a DNA molecule capable of specific hybridization under stringent conditions to a DNA molecule according to (a), and which encodes a protein that, when introduced into human osteoblasts, increases the ability of the osteoblasts to synthesize bone;
(iii) a DNA molecule having a nucleotide sequence which is degenerate as a result of the genetic code to the encoded protein amino acid sequence according to (ii) and which encodes a protein that, when introduced into osteoblast cells of humans, increases the ability of the cells to synthesize bone;
(iv) a DNA molecule capable of specific hybridization under stringent conditions to a DNA molecule according to (i), and which encodes a protein that, when introduced into endothelial cells of humans, increases the ability of the endothelial cells to form vascular tissue; and
(v) a DNA molecule having a nucleotide sequence which is degenerate as a result of the genetic code to the encoded protein amino acid sequence according to (iv), and which DNA molecule encodes a protein that, when introduced into endothelial cells of humans, increases the ability of the endothelial cells to form vascular tissue.

8. A method of regulating bone strength and mineralization by activating a bone density regulating transmembrane receptor encoded by an isolated DNA molecule useful for the diagnosis or therapy of osteoporosis, selected from the group consisting of:
(a) a DNA molecule having a nucleotide sequence encoding an amino acid sequence as shown in FIG. 2;
(b) a DNA molecule capable of specific hybridization under stringent conditions to a DNA molecule according to (a), and which encodes a protein that, when introduced into human osteoblasts, increases the ability of the osteoblasts to synthesize bone;
(c) a DNA molecule having a nucleotide sequence which is degenerate as a result of the genetic code to the encoded protein amino acid sequence according to (b) and which encodes a protein that, when introduced into osteoblast cells of humans, increases the ability of the cells to synthesize bone;
(d) a DNA molecule capable of specific hybridization under stringent conditions to a DNA molecule according to (a), and which encodes a protein that, when introduced into endothelial cells of humans, increases the ability of the endothelial cells to form vascular tissue; and
(e) a DNA molecule having a nucleotide sequence which is degenerate as a result of the genetic code to the encoded protein amino acid sequence according to (d), and which encodes a protein that, when introduced into endothelial cells of humans, increases the ability of the endothelial cells to form vascular tissue, wherein the method comprises a first step of providing a ligand of the transmembrane receptor and a second step of administering the provided ligand in a medically acceptable form.

9. A method as described in claim 8, wherein the ligand is selected from the group consisting of: RVRLASHLRKLRKRLLR (SEQ ID NO:83); RLTRKRGLKLA (SEQ ID NO:76); CRAKRNNFKSA (SEQ ID NO:77); LKWKS (SEQ ID NO:78); KIRVKAGETQKKVIFCSREKVSHL (SEQ ID NO:79); FIPLKPTVKMLERSNHVSRTEVSSNHV (SEQ ID NO:80); DKGMAPALRHLYKELMGPWN (SEQ ID NO:81) and DALKLAIDNALSIT (SEQ ID NO:82)

10. A method for determining bone strength and mineralization predisposition of a patient, the method comprising the steps of:
(a) providing a patient sample that contains nucleic acid that encodes the BSMR protein and;
(b) analysing the sample from step (a) to determine whether the contacting the sample of (a) with one or more second nucleic acid(s) having at least one sequence described by claim 1 under conditions of hybridization; and
(c) detecting the formation of a hybrid between the sample nucleic acid and the second nucleic acid.

11. A method for determining bone strength and mineralization predisposition of a patient from analysis of epitopes on the bone strength and mineralization regulator protein, the method comprising the steps of:
(a) providing a tissue or blood sample of the patient;
(b) contacting the sample from step (a) with at least one conjugate of an antibody or antibody fragment with a reporter molecule, wherein the antibody or antibody fragment recognizes one or more epitopes of the bone strength and mineralization regulator protein; and
(c) detecting the formation of a complex between the conjugate and protein within the sample.

12. A method as described in claim 11, wherein step (b) utilizes at least two different conjugates that bind to at least two different portions of the bone strength and mineralization regulator protein.

13. A method as described in claim 12, wherein one of the conjugates binds to an epitope within the amino terminal half of the protein and a second of the conjugates binds to an epitope outside the amino terminal half of the protein.

14. A method for enhancing bone strength and mineralization by a mammalian cell, the method comprising the step of introducing the expression vector of claim 5 into the cell.

15. A method of increasing bone strength and mineralization in a human subject, comprising the steps:
(a) providing a ligand and/or a protease of the bone strength and mineralization regulator protein; and
(b) administering the ligand and/or protease to the human subject.

16. A method as described in claim 15, wherein step (b) is carried out by injection or by oral delivery.

17. A method as described in claim 15, wherein the ligand is selected from the group consisting of: RVRLASHLRKLRKRLLR (SEQ ID NO:83); RLTRKRGLKLA (SEQ ID NO:76); CRAKRNNFKSA (SEQ ID NO:77); LKWKS (SEQ ID NO:78); KIRVKAGETQKKVIFCSREKVSHL (SEQ ID NO:79); FIPLKPTVKMLERSNHVSRTEVSSNHV (SEQ ID NO:80); DKGMAPALRHLYKELMGPWN (SEQ ID NO:81) and DALKLAIDNALSIT (SEQ ID NO:82).

18. A bone strength and mineralization regulator protein that lacks at least 59% of it sequence from the amino terminal end.

19. A method for discovering a pharmaceutical useful for regulating bone strength or mineralization, comprising the steps:
(a) providing a protein reagent comprising at least one ligand binding site of the BMSR protein;
(b) contacting the reagent from step (a) with a test substance; and
(c) detecting binding between the reagent from step (a) and the test substance, wherein binding indicates that the test substance affects bone strength or mineralization upon administration to a patient.

20. A method as described in claim 19, wherein the protein reagent of step (a) comprises 3 ligand binding sites from a single BMSR protein.

21. A method as described in claim 19, wherein the test substance is a peptide.

22. A composition that improves bone strength and mineralization, the composition comprising a BSMR effector that activates one or more biochemical pathways leading to increased bone mass.

23. The composition of claim 22, wherein the BSMR effector increases alkaline phosphatase activity of bone forming cells by at least 10% compared to cells that are not exposed to the BSMR effector.

24. The composition of claim 22, wherein the BSMR effector is selected from the group consisting of WNT1, WNT2, WNT2B/13, WNT3, WNT3A, WNT4, WNT5A, WNT5B, WNT6, WNT7A, WNT7B, WNT8A, WNT8B, WNT10A, WNT10B, WNT11, WNT14, WNT15, WNT16, the 36 kDa cysteine rich frizzled related protein Frzb—1, apolipoprotein, a cysteine rich protein from the CCN family7, Mus musculus FK506 binding protein 8, Mus musculus nuclear protein 95 (Np95); GLI-Kruppel family member GLI3, Mus musculus RAN binding protein 9, Mus musculus ISL1 transcription factor, Human signal-transducing guanine nucleotide-binding regulatory (G) protein beta subunit, Mus musculus, casein kinase II, Homo sapiens zinc finger protein 198, Mus musculus, eukaryotic translation elongation factor 2, M. musculus P311, Homo sapiens E2a—Pb×1-associated protein, Homo sapiens NADH dehydrogenase (ubiquinone) Fe—S protein 8, Human Smad anchor for receptor activation (SARA), Homo sapiens AMSH, and ATP6B2.

25. The composition of claim 24, wherein the BSMR effector is a WNT protein.

26. The composition of claim 22 that further comprises a second morphogenetic protein, wherein the second morphogenetic protein enhances the stimulation of bone mineralization.

27. The composition of claim 26, wherein the second morphogenetic protein is selected from the group consisting of bone morphogenetic protein, bone morphogenetic protein 2, bone morphogenetic protein 3, hedgehog protein, endothelial growth factor, and TGF—beta 26.

28. The composition of claim 22, wherein the BSMR effector is complexed with a targeting moiety that concentrates the effector at one or more bone producing regions after administration to a patient.

29. The composition of claim 28, wherein the targeting moiety is selected from the group consisting of a tetracycline, calcein, a bisphosphonate complex, polyaspartic acid, polyglutamic acid, an aminophosphosugar, a peptide known to be associated with the mineral phase of bone, osteonectin, bone sialoprotein, osteopontin, a bone specific antibody, a binding site fragment of a bone specific antibody, and a protein having a bone mineral binding domain.

29. A method of treating osteoporosis in a human patient, comprising:
(a) providing a composition that comprises a BSMR effector; and
(b) administering a quantity of the composition from step (a) to the patient that is sufficient to increase alkaline phosphatase activity of bone forming cells.

30. The method of claim 29, wherein the BSMR effector is selected from the group consisting of WNT1, WNT2, WNT2B/13, WNT3, WNT3A, WNT4, WNT5A, WNT5B, WNT6, WNT7A, WNT7B, WNT8A, WNT8B, WNT10A, WNT10B, WNT11, WNT14, WNT15, WNT16, the 36 kDa cysteine rich frizzled related protein Frzb—1, apolipoprotein, a cysteine rich protein from the CCN family7, Mus musculus FK506 binding protein 8, Mus musculus nuclear protein 95 (Np95); GLI-Kruppel family member GLI3, Mus musculus RAN binding protein 9, Mus musculus ISL1 transcription factor, Human signal-transducing guanine nucleotide-binding regulatory (G) protein beta subunit, Mus musculus, casein kinase II, Homo sapiens zinc finger protein 198, Mus musculus, eukaryotic translation elongation factor 2, M. musculus P311, Homo sapiens E2a—Pb×1-associated protein, Homo sapiens NADH dehydrogenase (ubiquinone) Fe—S protein 8, Human Smad anchor for receptor activation (SARA), Homo sapiens AMSH, and ATP6B2.

31. The method of claim 30, wherein the BSMR effector is a WNT protein.

32. The method of claim 29, wherein the BSMR effector is complexed with a targeting moiety that concentrates the effector at one or more bone producing regions after administration to a patient.

33. The method of claim 32, wherein the targeting moiety is selected from the group consisting of a tetracycline, calcein, a bisphosphonate complex, polyaspartic acid, polyglutamic acid, an aminophosphosugar, a peptide known to be associated with the mineral phase of bone, osteonectin, bone sialoprotein, osteopontin, a bone specific antibody, a binding site fragment of a bone specific antibody, and a protein having a bone mineral binding domain.

34. The method of claim 29, further comprising an additional step of administering a second morphogenetic protein at least 24 hours prior to step (b).

35. The method of claim 34, wherein the second morphogenetic protein is selected from the group consisting of bone morphogenetic protein, bone morphogenetic protein 2, bone morphogenetic protein 3, hedgehog protein, endothelial growth factor, and TGF—beta 26.
